(19) United States
(12) Patent Application Publication
(10) Pub. No.: US 2015/0152503 A1
(43) Pub. Date: Jun. 4, 2015

(54) MICRORNAS FOR PREDICTION OF TREATMENT EFFICACY AND PROGNOSIS OF CANCER PATIENTS

(71) Applicant: Herlev Hospital, Herlev (DK)

(72) Inventors: Mogens Karsbøl Boisen, Allerod (DK); Julia Sidenius Johansen, Frederiksberg (DK)

(21) Appl. No.: 14/372,165

(22) PCT Filed: Jan. 16, 2013
(86) PCT No.: PCT/DK2013/050015

§ 371 Date: Jul. 14, 2014

(30) Foreign Application Priority Data
Jan. 16, 2012 (DK) PA 2012 70025

(51) Int. Cl. C12Q 1/68

(52) U.S. Cl. CPC C12Q 1/6886; C12Q 2600/178; C12Q 2600/106; C12Q 2600/158

(57) ABSTRACT

The present invention lies within the field of personalised medicine. More particular the invention relates to biomarkers useful for predicting treatment efficacy and prognosis in cancer patients. Thus the invention provides microRNAs (miRNAs) which are useful for predicting efficacy of anti-angiogenic treatment. In particular, miR-664 for the prediction of response to bevacizumab in colorectal cancer of sigmoid colon or rectum.

### FIELD OF INVENTION

The present invention lies within the field of personalised medicine. More particular the invention relates to biomarkers useful for predicting treatment efficacy and prognosis in cancer patients. Thus the invention provides microRNAs (miRNAs) which are useful for predicting efficacy of anti-angiogenic treatment.

### BACKGROUND OF INVENTION

Colorectal cancer (CRC) is the 3^{rd} most common cause of cancer death in the United States (˜51.000 deaths/year) and Europe (˜207.500 deaths/year). In Denmark 2.000 patients die each year because of CRC. In the past decade the survival of patients with metastatic CRC has improved due to new combinations of chemotherapy, including 5-fluorouracil, irinotecan, and oxaliplatin. The introduction of new targeted therapy directed against either the vascular endothelial growth factor (VEGF) or the epidermal growth factor receptor (EGFR) has further increased survival and response rates in some patients. It is not known why only half of the patients benefit from chemotherapy and only a small subset of the patients benefit from treatment with an antibody targeting VEGF (bevacizumab, Avastin®) or an antibody targeting EGFR (cetuximab, Erbitux®).

The high cost of biologic treatment has become an increasing problem in cancer care and the health care systems all over the world.

### SUMMARY OF INVENTION

This has raised a demand for identification of patients who will benefit from biological therapy and in particular a demand for identification of patients who will benefit from treatment with bevacizumab or other therapeutics targeting angiogenesis.

Thus the present invention provides methods for predicting the efficacy of an anti-angiogenic treatment in an individual suffering from cancer, said method comprising the steps of

i) providing a sample comprising cancer cells from said individual;
ii) determining the expression level of a combination of miRNAs in said sample wherein the combination comprises
a) Optionally miR-664; and
b) One or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

Thus the present invention also provides methods for predicting the efficacy of an anti-angiogenic treatment in an individual suffering from cancer, said method comprising the steps of

i) providing a sample comprising cancer cells from said individual;
ii) determining the expression level of a combination of miRNAs in said sample, wherein the combination comprises
a) one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
b) one or more miRNAs selected from the group consisting of miR-382, miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c.
   wherein the miRNA expression level, and/or an aberrant miRNA expression level of at least one of said miRNAs is indicative of the efficacy of an anti-angiogenic treatment of said individual.

The invention also provides methods for predicting the efficacy of a chemotherapeutic treatment in an individual suffering from cancer, said method comprising the steps of

i) providing a sample comprising cancer cells from said individual;
ii) determining the expression level of a combination of miRNAs in said sample, wherein the combination comprises
a) one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
b) one or more miRNAs selected from the group consisting of miR-382, miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c.
   wherein the miRNA expression level, and/or an aberrant miRNA expression level of at least one of said miRNAs is indicative of the efficacy of an anti-angiogenic treatment of said individual.

The invention also provides methods of treatment of cancer in an individual in need thereof, said methods comprising the steps of
i) Predicting the efficacy of an anti-angiogenic treatment in said individual by the methods described herein
ii) Administering a therapeutically effective amount of an anti-angiogenic treatment to said individual, provided the prediction of said efficacy is good
   thereby treating cancer in said individual

The invention also provides methods of treatment of cancer in an individual in need thereof, said methods comprising the steps of
i) Predicting the efficacy of a chemotherapeutic treatment by the methods described herein
ii) Administering a therapeutically effective amount of a chemotherapeutic treatment to said individual, provided the prediction of said efficacy is good
   thereby treating cancer in said individual

The invention also provides miRNA classifiers for characterising a sample obtained from an individual, wherein said miRNA classifier comprises or consists of a combination of miRNAs comprising
a) one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
b) one or more miRNAs selected from the group consisting of miR-382, miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c,

The invention furthermore provides a device for measuring the expression level of at least one miRNA in a sample, wherein said device comprises or consists of at least one probe or primer set for a combination of miRNAs comprising
a) one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
b) one or more miRNAs selected from the group consisting of miR-382, miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c.
   wherein said device is used for characterising a sample.

In relation to all methods, classifiers and devices described herein it is preferred that the miRNA selected under a) is different to the miRNA selected under b). Furthermore, in preferred embodiments the combination of miRNAs comprises miR-382 in addition to a miRNA selected under a) and a miRNA selected under b), wherein said miRNAs selected under a) and b) are not miR-382.

### DESCRIPTION OF DRAWINGS

FIG. 1 shows the estimated time to disease progression (TTP) based on the predictive value of miR-22 expression in the multivariate model.

FIG. 2 shows the estimated overall survival (OS) based on the predictive value of miR-382 expression in the multivariate model.

FIG. 3 shows a Kaplan-Meier plot of OS for patients with values above and below median for a prognostic index based on miR-382 and miR-196b expression.

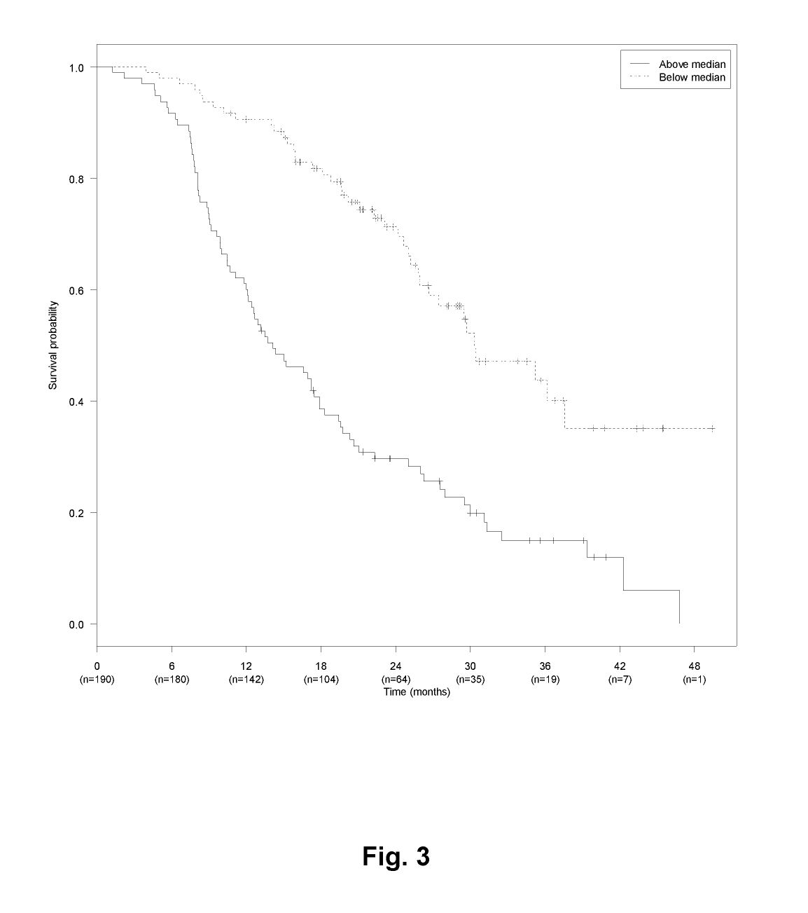

FIG. 4 shows a Kaplan-Meier plot of TTP for patients with values above and below median for a prognostic index based on miR-22, miR-29b, miR-145*, and miR-193b* expression.

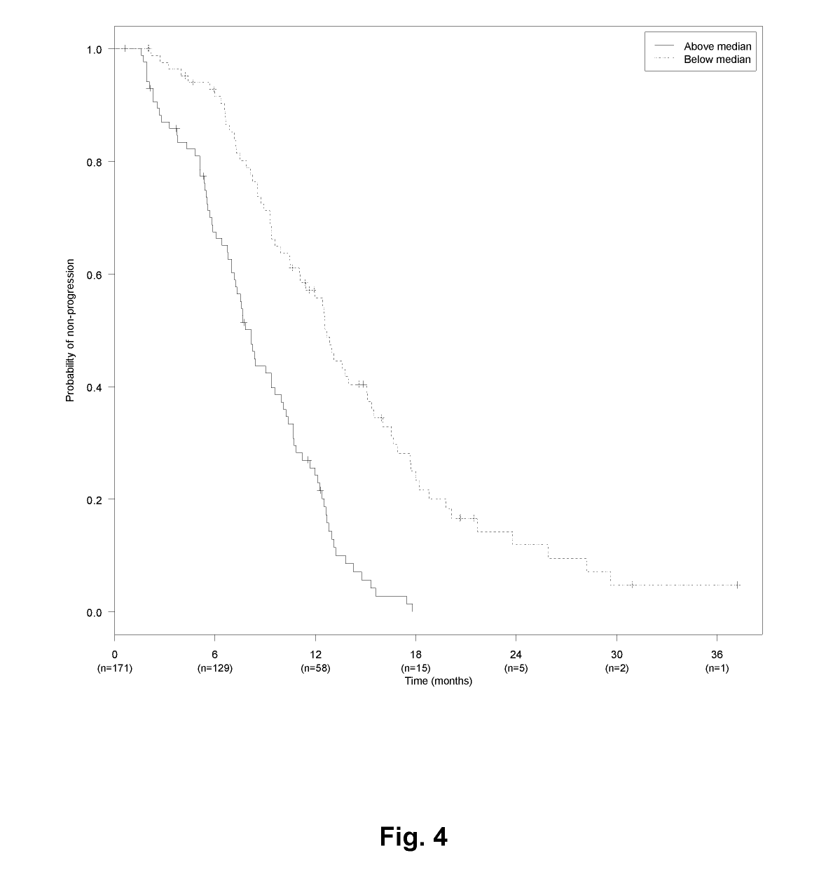

FIG. 5 shows Kaplan Meier plots of OS by quartiles of raw miR-664 expression for patients with metastatic colorectal cancer originating in the sigmoid colon, recto-sigmoid colon, and rectum who were treated with CapOx with or without bevacizumab.

FIG. 6 shows Kaplan Meier plots of OS by quartiles of raw miR-664 expression for patients with metastatic colorectal cancer originating in the caecum, ascending colon, right flexure, transverse colon, left flexure, and descending colon who were treated with CapOx with or without bevacizumab.

FIG. 7 shows response rates and Kaplan Meier plots of OS for patients with the highest and lowest expression values of miR-664 stratified by primary tumor location group (15 patients in each of 4 groups).

### DETAILED DESCRIPTION OF THE INVENTION

### Definitions

A ‘biomarker’ may be defined as a biological molecule found in tissues or body fluids that is an indicator of a normal or abnormal process, or of a condition or disease. A biomarker may be used to foresee how well the body responds to a treatment for a disease or condition, or may be used to associate a certain disease or condition to a certain value of said biomarker found in e.g. a tissue sample. Biomarkers are also called molecular markers and signature molecules.

‘Collection media’ as used herein denotes any solution suitable for collecting, storing or extracting a sample for immediate or later retrieval of RNA from said sample.

‘Deregulated’ means that the expression of a miRNA is altered from its normal baseline levels; comprising both up- and down-regulated.

The term “Individual” refers to vertebrates, in particular members of the mammalian species, preferably primates including humans. As used herein, ‘subject’ and ‘individual’ may be used interchangeably.

The term “Kit of parts” as used herein provides a device for measuring the expression level of at least one miRNA as identified herein, and at least one additional component. The additional component may be used simultaneously, sequentially or separately with the device. The additional component may in one embodiment be means for extracting RNA, such as miRNA, from a sample; reagents for performing microarray analysis, reagents for performing quantitative real time polymerase chain reaction (qPCR) analysis and/or instructions for use of the device and/or additional components.

A microRNA is a short RNA. MicroRNAs may also be denoted miRNA or miR herein. Preferably a miRNA to be used with the present invention is 19-25 nucleotides in length and consists of non-protein-coding RNA. Mature miRNAs may exert, together with the RNA-induced silencing complex, a regulatory effect on protein synthesis at the post-transcriptional level. More than 1500 human miRNA sequences have been discovered to date and their names and sequences are available from the miRBase database (http://www.mirbase.org).

The term “natural nucleotide” or “nucleotide” refers to any of the four deoxyribonucleotides, dA, dG, dT, and dC (constituents of DNA), and the four ribonucleotides, A, G, U, and C (constituents of RNA). Each natural nucleotide comprises or essentially consists of a sugar moiety (ribose or deoxyribose), a phosphate moiety, and a natural/standard base moiety.

As used herein, “nucleic acid” or “nucleic acid molecule” refers to polynucleotides, such as deoxyribonucleic acid (DNA) or ribonucleic acid (RNA), oligonucleotides, fragments generated by the polymerase chain reaction (PCR) or ligation chain reaction, and fragments generated by any of ligation, scission, endonuclease action, and exonuclease action. Nucleic acid molecules can be composed of monomers that are naturally-occurring nucleotides (such as DNA and RNA), or analogs of naturally-occurring nucleotides (e.g. alpha-enantiomeric forms of naturally-occurring nucleotides), or a combination of both. Modified nucleotides can have alterations in sugar moieties and/or in pyrimidine or purine base moieties. Sugar modifications include, for example, replacement of one or more hydroxyl groups with halogens, alkyl groups, amines, and azido groups, or sugars can be functionalized as ethers or esters. Moreover, the entire sugar moiety can be replaced with sterically and electronically similar structures, such as aza-sugars and carbocyclic sugar analogs. Examples of modifications in a base moiety include alkylated purines and pyrimidines, acylated purines or pyrimidines, or other well-known heterocyclic substitutes. Nucleic acid monomers can be linked by phosphodiester bonds or analogs of such linkages. Analogs of phosphodiester linkages include phosphorothioate, phosphorodithioate, phosphoroselenoate, phosphorodiselenoate, phosphoroanilothioate, phosphoranilidate, phosphoramidate, and the like. The term “nucleic acid molecule” also includes e.g. so-called “peptide nucleic acids,” which comprise naturally-occurring or modified nucleic acid bases attached to a polyamide backbone. Nucleic acids can be either single stranded or double stranded. In an aspect of the present invention, ‘nucleic acid’ is meant to comprise antisense oligonucleotides (ASO), small inhibitory RNAs (sRNA), short hairpin RNA (shRNA) and microRNA (miRNA).

A “polypeptide” or “protein” is a polymer of amino acid residues preferably joined exclusively by peptide bonds, whether produced naturally or synthetically. The term “polypeptide” as used herein covers proteins, peptides and polypeptides, wherein said proteins, peptides or polypeptides may or may not have been post-translationally modified. Post-translational modification may for example be phosphorylation, methylation and glycosylation.

A ‘primer’ as used herein refers to a short nucleic acid, typically DNA, which may be used in an amplification procedure, such as in PCR.

A ‘probe’ as used herein refers to a hybridization probe. A hybridization probe is a (single-stranded) fragment of DNA or RNA of variable length (usually 100-1000 bases long), which is used in DNA or RNA samples to detect the presence of nucleotide sequences (the DNA target) that are complementary to the sequence in the probe. The probe thereby hybridizes to single-stranded nucleic acid (DNA or RNA) whose base sequence allows probe-target base pairing due to complementarity between the probe and target. To detect hybridization of the probe to its target sequence, the probe is tagged (or labelled) with a molecular marker of either radioactive or fluorescent molecules. DNA sequences or RNA transcripts that have moderate to high sequence similarity to the probe are then detected by visualizing the hybridized probe. Hybridization probes used in DNA microarrays refer to DNA covalently attached to an inert surface, such as coated glass slides or gene chips, and to which a mobile cDNA target is hybridized.

Due to the imprecision of standard analytical methods, molecular weights and lengths of polymers are understood to be approximate values. When such a value is expressed as “about” X or “approximately” X, the stated value of X will be understood to be accurate to +/−20%, such as +/−10%, for example+/−5%.

### DETAILED DESCRIPTION OF THE INVENTION

### MicroRNA

MicroRNAs (miRNA) are single-stranded RNA molecules of about 19-25 nucleotides in length, which regulate gene expression. miRNAs are either expressed from non-protein-coding transcripts or mostly expressed from protein coding transcripts. They are processed from primary transcripts known as pri-miRNA to shorter stem-loop structures called pre-miRNA and finally to functional mature miRNA. Mature miRNA molecules are partially complementary to one or more messenger RNA (mRNA) molecules, and their main function is to inhibit gene expression. This may occur by preventing mRNA translation or increasing mRNA turnover/degradation.

The transcripts encoding miRNAs are much longer than the processed mature miRNA molecule; miRNAs are first transcribed as primary transcripts or pri-miRNA with a cap and poly-A tail by RNA polymerase II and processed to short, 70-nucleotide stem-loop structures known as pre-miRNA in the cell nucleus. This processing is performed in animals (including humans) by a protein complex known as the Microprocessor complex, consisting of the ribonuclease III Drosha and the double-stranded RNA binding protein Pasha. These pre-miRNAs are then exported to the cytoplasm by Exportin-5/Ran-GTP and processed to mature miRNAs by interaction with the ribonuclease III Dicer and separation of the miRNA duplexes. The mature single-stranded miRNA is incorporated into a RNA-induced silencing complex (RISC)-like ribonucleoprotein particle (miRNP). The RISC complex is responsible for the gene silencing observed due to miRNA expression and RNA interference. The pathway is different for miRNAs derived from intronic stem-loops; these are processed by Dicer but not by Drosha.

When Dicer cleaves the pre-miRNA stem-loop, two complementary short RNA molecules are formed, but only one is integrated into the RISC complex. This strand is known as the guide strand and is selected by the argonaute protein, the catalytically active RNase in the RISC complex, on the basis of the stability of the 5′ end. The remaining strand, known as the anti-guide or passenger strand, is degraded as a RISC complex substrate. After integration into the active RISC complex, miRNAs base pair with their complementary mRNA molecules. This may induce mRNA degradation by argonaute proteins, the catalytically active members of the RISC complex, or it may inhibit mRNA translation into proteins without mRNA degradation.

The function of miRNAs appears to be mainly in gene regulation. For that purpose, an miRNA is (partly) complementary to a part of one or more mRNAs. Animal (including human) miRNAs are usually complementary to a site in the 3′ UTR. The annealing of the miRNA to the mRNA then inhibits protein translation, and sometimes facilitates cleavage of the mRNA (depending on the degree of complementarity). In such cases, the formation of the double-stranded RNA through the binding of the miRNA to mRNA inhibits the mRNA transcript through a process similar to RNA interference (RNAi).

Furthermore, miRNAs may regulate gene expression post-transcriptionally at the level of translational inhibition at P-bodies. These are regions within the cytoplasm consisting of many enzymes involved in mRNA turnover; P bodies are likely the site of miRNA action, as miRNA-targeted mRNAs are recruited to P bodies and degraded or sequestered from the translational machinery. In other cases it is believed that the miRNA complex blocks the protein translation machinery or otherwise prevents protein translation without causing the mRNA to be degraded. miRNAs may also target methylation of genomic sites which correspond to targeted mRNAs. miRNAs function in association with a complement of proteins collectively termed the miRNP (miRNA ribonucleoprotein complex).

Under a standard nomenclature system, miRNA names are assigned to experimentally confirmed miRNAs before publication of their discovery. The prefix “mir” is followed by a dash and a number, the latter often indicating order of naming. For example, mir-22 was named and likely discovered prior to mir-382. The uncapitalized “mir-” refers to the pre-miRNA, while a capitalized “miR-” refers to the mature form. miRNAs with nearly identical sequences bar one or two nucleotides are annotated with an additional lower case letter. For example, miR-129a would be closely related to miR-129b. miRNAs that are 100% identical but are encoded at different places in the genome are indicated with additional dash-number suffix. Species of origin is designated with a three-letter prefix, e.g., hsa-miR-123 would be from human (Homo sapiens) and oar-miR-123 would be a sheep (Ovis aries) miRNA. Other common prefixes include ‘v’ for viral (miRNA encoded by a viral genome) and ‘d’ for Drosophila miRNA. MicroRNAs originating from the 3′ or 5′ end of a pre-miRNA are denoted with a -3p or -5p suffix. (In the past, this distinction was also made with ‘s’ (sense) and ‘as’ (antisense)).

An asterisk following the name indicates that the miRNA is an anti-miRNA to the miRNA without an asterisk (e.g. miR-214* is an anti-miRNA to miR-214). When relative expression levels are known, an asterisk following the name indicates a miRNA expressed at low levels relative to the miRNA in the opposite arm of a hairpin. For example, miR-214 and miR-214* would share a pre-miRNA hairpin, but relatively more miR-214 would be found in the cell.

As used herein, it is understood that ‘rniR-’ and ‘hsa-miR’ is used interchangeably; the results of the present invention are obtained from human samples and human miRNAs are examined.

miRBase is the central online repository for microRNA (miRNA) nomenclature, sequence data, annotation and target prediction, and may be accessed via http://www.mirbase.org/. The miRNA names used herein throughout can be accessed via this link, and specifics retrieved. See also Griffiths-Jones et al, “miRBase: tools for microRNA genomics”, Nucleic Acids Research, 2008, Vol. 36, Database issue D154-D158.

The names of the miRNAs herein are as used by TaqMan® Human MicroRNA A Cards v2.0 and B Cards v3.0 (Part Number 4400238, Applied Biosystems). The sequences corresponding to the various names are those available in the miRBase database version 18, November 2011, www.mirbase.org (homo sapiens).

### Biomarker

A biomarker, or biological marker, is in general a substance used as an indicator of a biological state. It is a characteristic that is objectively measured and evaluated as an indicator of normal biological processes, pathogenic processes, or pharmacologic responses to a therapeutic intervention.

More specifically, a biomarker can indicate a change in expression or state of e.g. a miRNA, a gene, a protein or a metabolite which correlates with e.g. the risk of progression of a disease, with the susceptibility of the disease to a given treatment, or with the risk of death.

A biomarker, such as a miRNA biomarker, may be correlated to a predicted efficacy of anti-angiogenic treatment and/or a chemotherapeutic treatment based on differences in miRNA expression levels in samples from a patient and a predetermined control level.

The predetermined control level may be the average level of expression in healthy controls.

However, more preferably the control level may be the average level of expression in patients with similar disease, where said other patients have been treated with an anti-angiogenic treatment and have a long time to disease progression (TTP) and/or a long time to death (i.e. long overall survival time (OS)) from time of starting an anti-angiogenic treatment. Such patients may also be referred to as patients with good efficacy of anti-angiogenic treatment. Preferably the average is made from at least 25, such as at least 50 patients or more preferably 100 patients. Preferably long TTP is at least 12 months and long OS is survival for at least 24 months or more preferably for at least 30 months.

In embodiments of the invention where the predetermined control level is the average level of expression in patients with good efficacy of anti-angiogenic treatment then if a certain miRNA biomarker is found to be deregulated in a sample as compared to a predetermined control level, the sample has a low probability of being associated with a good efficacy of anti-angiogenic treatment. Vice versa if a certain miRNA biomarker in a sample is found to have an expression level which is close to the predetermined control level, the sample has a high probability of being associated with a good efficacy of anti-angiogenic treatment.

The predetermined control level may also be the average level of expression in patients with similar disease, where said other patients have been treated with an anti-angiogenic treatment and have a short time to disease progression (TTP) and/or a short time to death, i.e. a short overall survival time (OS) from time of starting an anti-angiogenic treatment. Such patients may also be referred to as patients with little or no efficacy of anti-angiogenic treatment. Preferably the average is made from at least 25, such as at least 50 patients or more preferably 100 patients. Preferably short TTP is at the most 6 months and short OS is survival for at the most 12 months.

In embodiments of the invention where the predetermined control level is the average level of expression in patients with no or little efficacy of anti-angiogenic treatment then if a certain miRNA biomarker is found to be deregulated in a sample as compared to a predetermined control level, the sample has a high probability of being associated with a good efficacy of anti-angiogenic treatment.

### MiRNA Biomarkers of the Present Invention

The miRNA biomarkers of the present invention are useful for predicting the efficacy of an anti-angiogenic treatment and/or a chemotherapeutic treatment in an individual suffering from cancer, and in particular in an individual suffering from cancer in colon or rectum.

In a preferred embodiment of the present invention the miRNA biomarkers are useful for predicting the efficacy of an anti-angiogenic treatment and/or a chemotherapeutic treatment in an individual suffering from cancer in the sigmoid colon, the rectum and/or the recto-sigmoid colon, and in particular in an individual suffering from cancer, where the primary tumour is positioned in the sigmoid colon, the rectum and/or the recto-sigmoid colon.

The miRNA biomarkers to be used with the present invention is preferably a combination of one or more miRNAs selected from the group consisting of miR-664, miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

The miRNA biomarkers to be used with the present invention is preferably a combination of biomarker, which consists of the following miRNAs:
a) optionally miR-664; and
b) one or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

In another embodiment the miRNA biomarkers to be used with the present invention is preferably a combination of biomarkers, which consists of the following miRNAs:
c) optionally miR-382; and
d) one or more miRNAs selected from the group consisting of miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
e) one or more miRNAs selected from the group consisting of miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c.

The miRNAs described under d) are mainly associated with TTP and the miRNAs described under e) are mainly associated with OS. Thus the miRNAs described under d) are particularly useful for predicting efficacy of an anti-angiogenic treatment in terms of increased TTP, whereas the miRNAs described under e) are particularly useful for predicting efficacy of an anti-angiogenic treatment in terms of increase OS. It is preferred that at least one miR selected under d) is different from at least one miRNA selected under e).

In another embodiment of the invention the miRNA biomarkers are preferably a combination of biomarkers, which consists of the following miRNAs:
f) miR-382; and
g) one or more miRNAs selected from the group consisting of miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
h) one or more miR selected from the group consisting of miR-592, miR-196b, miR-338-3p, miR-133b, miR-15a, miR-497, miR-552, miR-181a, miR-141, miR-545, miR-21*, miR-452, miR-501-5p, and miR-29c.

In another embodiment of the invention the miRNA biomarkers are preferably a combination of biomarkers, which consists of the following miRNAs:
i) one or more miRs selected from the group consisting of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p, and miR-625; and
j) one or more miR selected from the group consisting of miR-196b, miR-592, miR-545, miR-15a, miR-455-5p, miR-338-3p, miR-19b, miR-660, miR-148a, miR-449a, miR-106b, miR-141, miR-18b, miR-379, miR-552, miR-29c, miR-181a, miR-193a-3p, and miR-636.

In another embodiment of the invention the miRNA biomarkers are preferably selected from the group consisting of the following miRNAs:
k) one or more miRs selected from the group consisting of miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

In yet another embodiment of the invention the miRNA biomarkers to be used with the present invention are preferably at least two miRNAs, which are selected from the group consisting of the miRNAs mentioned in Tables 1, 2, 3 and 4, and more preferably at least two miRNAs selected from the group consisting of the miRNAs mentioned in Tables 1, 2, 3 and 4 having a p-value of less than 0.020, more preferably having a p-value of less than 0.015, even more preferably having a p-value of less than 0.013, for example having a p-value of less than 0.010, such as having a p-value of less than 0.008, for example having a p-value of less than 0.007.

It is contemplated that the expression level of at least one of said miRNAs in one embodiment is measured in a sample from an individual suffering from cancer, and said miRNA expression level as compared to a predetermined control level, wherein the predetermined control level is the level in patients with known good efficacy or no/little efficacy of an anti-angiogenic treatment and the miRNA expression level in the patient suffering from cancer is then associated with a specific predicted efficacy of an anti-angiogenic treatment.

In a particular embodiment, the difference between the expression levels of two miRNAs is calculated; wherein said difference in expression levels between said two miRNAs may be used to correlate said difference in miRNA expression level to a certain predicted efficacy. Said difference may thus be a relative difference.

In one embodiment, said biomarkers are used in combination (′simple combination′); i.e. the expression level of at least the three miRNAs according to c) to e) or f) to h) immediately herein above are all used in combination to distinguish or separate the efficacy of an anti-angiogenic treatment.

It is contemplated according to the present invention that a similar expression level of at least the three miRNAs according to or c) to e) or f) to h) immediately herein above compared to a predetermined control level of patients with good efficacy of anti-angiogenic treatment is indicative of that anti-angiogenic treatment will be effective, and in particular it is indicative of that anti-angiogenic treatment may lead to increased TTP and/or increased OS.

It is also contemplated according to the present invention that a difference in expression level of at least the three miRNAs according to c) to e) or f) to h) immediately herein above compared to a predetermined control level of patients with no or little efficacy of an anti-angiogenic treatment is indicative of that anti-angiogenic treatment will be effective, and in particular it is indicative of that anti-angiogenic treatment may lead to increased TTP and/or increased OS.

In one embodiment, miR-193b* as a biomarker is claimed only in combination with another miRNA as a biomarker for predicting the efficacy of an anti-angiogenic treatment in terms of TTP and/or OS, wherein at least one of said other miRNAs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-196b, miR-204, miR-214*, miR-338-3p, miR-365, miR-382, miR-449a, miR-449b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, miR-145* as a biomarker is claimed only in combination with another miR as a biomarker for predicting the efficacy of an anti-angiogenic treatment in terms of TTP and/or OS, wherein at least one of said other miRs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-148a, miR-155, miR-181a, miR-185, miR-193b*, miR-196b, miR-204, miR-214*, miR-338-3p, miR-365, miR-382, miR-449a, miR-449b, miR-455-5p, miR-497, miR-501-5p, miR-545, miR-552, miR-592, miR-664 and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, miR-29b as a biomarker is claimed only in combination with another miR as a biomarker for predicting the efficacy of an anti-angiogenic treatment in terms of TTP and/or OS, wherein at least one of said other miRs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664 and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, miR-22 as a biomarker is claimed only in combination with another miR as a biomarker for predicting the efficacy of an anti-angiogenic treatment in terms of TTP and/or OS, wherein at least one of said other miRs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449b, miR-455-5p miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, miR-382 as a biomarker is claimed only in combination with another miR as a biomarker for predicting the efficacy of an anti-angiogenic treatment in terms of TTP and/or OS, wherein at least one of said other miRs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-449a, miR-449b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, miR-196b as a biomarker is claimed only in combination with another miR as a biomarker for predicting the efficacy of an anti-angiogenic treatment in terms of TTP and/or OS, wherein at least one of said other miRs may be selected from the group consisting of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-17* as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-29a* as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-185 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-204 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-214* as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-365 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-497 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-501-5p as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-664 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-1251 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-15a as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-148a as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-155 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-204 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. 58. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-214* as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-338-3p as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-449a as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-449b as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-455-5p as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-545 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention miR-592 as a biomarker is claimed only in combination with another miRNA as a biomarker for predicting the efficacy of an anti-angiogenic treatment in terms of TTP and/or OS, wherein at least one of said other miRNAs is selected from the group of miRNAs mentioned in Tables 1, 2, 3 and 4, preferably from the group of miRNAs mentioned in Tables 5 and 6, even more preferably from the group consisting of miR-1, miR-15a, miR-17*, miR-22, miR-29a*, miR-29b, miR-145*, miR-148a, miR-155, miR-181a, miR-185, miR-193b*, miR-196b, miR-204, miR-214*, miR-338-3p, miR-365, miR-382, miR-449a, miR-449 b, miR-455-5p, miR-497, miR-501-5p, miR-545, miR-552, miR-592, miR-664, and miR-1251. 67. Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-17* and said other miRNA is selected from the group of miRNAs mentioned in Tables 1, 2, 3 and 4, preferably from the group of miRNAs mentioned in Tables 5 and 6, even more preferably from the group consisting of miR-155, miR-185, miR-196b, miR-204, miR-214*, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-155 and said other miRNA is selected from the group of miRNAs mentioned in Tables 1, 2, 3 and 4, preferably from the group of miRNAs mentioned in Tables 5 and 6, even more preferably from the group consisting of miR-17*, miR-185, miR-196b, miR-204, miR-214*, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-185 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-196b, miR-204, miR-214*, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-196b and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-204, miR-214*, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-17* and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-214*, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-214 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-204, miR-382, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-382 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-204, miR-214*, miR-455, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-455 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-204, miR-214*, miR-382, miR-545 and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-545 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-204, miR-214*, miR-382, miR-455, and miR-664. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment of the invention at least two miRNAs are used for predicting the efficacy of an anti-angiogenic treatment in terms of TTP and/or OS, wherein one miRNA is miR-664 and said other miRNA is selected from the group of miRNAs mentioned in Tables 1, 2, 3 and 4, preferably from the group of miRNAs mentioned in Tables 5 and 6, even more preferably from the group consisting of miR-17*, miR-155, miR-185, miR-196b, miR-204, miR-214*, miR-382, miR-455, and miR-545. This is in particular relevant in embodiments of the invention where the individual is an individual suffering from colorectal cancer, wherein the primary tymor is located in the sigmoid colon, the rectum and/or the recto-sigmoid colon.

Thus, for example if said at least two miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In a further aspect, the present invention discloses miRNA biomarkers that are significantly differentially expressed between cancer patients, for whom anti-angiogenic treatment has good efficacy vs. cancer patients for whom anti-angiogenic treatment has little or no efficacy.

In a further aspect, the present invention discloses miRNA biomarkers that are significantly differentially expressed between cancer patients, for whom chemotherapeutic treatment has good efficacy vs. cancer patients for whom chemotherapeutic treatment has little or no efficacy.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more, preferably at least two, more preferably at least 3, even more preferably at least 4 miRNAs selected from the group of miRNAs mentioned in Tables 1, 2, 3 and 4. Said miRNAS may preferably be selected from the group of miRNAs mentioned in Tables 3 and 4. More preferably said miRNAs may be selected from the group consisting of miRNAs mentioned in Tables 5 and 6. Even more preferably said miRNAs may be at least one miRNA selected from the group consisting of miRNAs mentioned in Table 5 and at least one miRNA selected from the group consisting of miRs mentioned in Table 6.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group consisting of miR-382, miR-370, miR-193b*, miR-22, miR-497, miR-29c*, miR-145*, miR-501-5-p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100, miR-151-3p, miR-592, miR-196b, miRmiR-338-3p, miR-133b, miR-15a, miR-497, miR-552, miR-181a, miR-141, miR-545, miR-21*, miR-452, and miR-29c.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100, miR-151-3p, miR-19b-1*, miR-664, miR-1285, miR-155, miR-532-3p, miR-1, miR-1227, miR-365, miR-145 and miR-625.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group consisting of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p, miR-625, miR-196b, miR-592, miR-545, miR-15a, miR-455-5p, miR-338-3p, miR-19b, miR-660, miR-148a, miR-449a, miR-106b, miR-141, miR-18b, miR-379, miR-552, miR-29c, miR-181a, miR-193a-3p, and miR-636.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group of miR-22, miR-1, miR-17*, miR-29a*, miR-29b, miR-145*, miR-185, miR-193b*, miR-204, miR-214*, miR-365, miR-382, miR-497, miR-501-5p, miR-664, miR-1251, miR-15a, miR-148a, miR-155, miR-181a, miR-196b, miR-338-3p, miR-449a, miR-449 b, miR-455-5p, miR-545, miR-552 and miR-592.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100, miR-151-3p, miR-592, miR-196b, miRmiR-338-3p, miR-133b, miR-15a, miR-552, miR-181a, miR-141, miR-545, miR-21*, miR-452, and miR-29c, miR-19b-1*, miR-664, miR-1285, miR-155, miR-532-3p, miR-1, miR-1227, miR-365, miR-145 and miR-625, miR-19b, miR-148a, miR-449a, miR-106b, miR-18b, miR-379, miR-193a-3p, miR-636, miR-1251, miR-449 b, miR-449, miR-455, miR-501.

Thus, for example if the one or more of said miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group of miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group of miR-17*, miR-155, miR-185, miR-196b, miR-204, miR-214*, miR-382, miR-455, miR-545, and miR-664.

Thus, for example if the one or more miRNAs in a sample is found to have an expression level which is close to the predetermined control level, wherein the predetermined control level is the level in patients with known good efficacy of an anti-angiogenic treatment, the sample has a high probability of being associated with a good efficacy of anti-angiogenic treatment.

In one embodiment, said miRNA biomarkers may be used to distinguish between cancer patients, for whom anti-angiogenic treatment increases TTP vs. cancer patients for whom anti-angiogenic treatment has little or no effect on TTP and said biomarkers may comprise one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p.

In one embodiment, said miRNA biomarkers may be used to distinguish between cancer patients, for whom anti-angiogenic treatment increases TTP vs. cancer patients for whom anti-angiogenic treatment has little or no effect on TTP, and said biomarkers may comprise one or more miRNAs selected from the group of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p, and miR-625.

In one embodiment, said miRNA biomarkers may be used to distinguish between cancer patients, for whom anti-angiogenic treatment increases TTP vs. cancer patients for whom anti-angiogenic treatment has little or no effect on TTP and said biomarkers may comprise one or more miRNAs selected from the group consisting of miR-1, miR-17*, miR-22, miR-29a*, miR-29b, miR-145*, miR-185, miR-193b*, miR-204, miR-214*, miR-365, miR-382, miR-497, miR-501-5p, miR-664, and miR-1251.

In one embodiment, said miRNA biomarkers may be used to distinguish between cancer patients, for whom anti-angiogenic treatment increases TTP vs. cancer patients for whom anti-angiogenic treatment has little or no effect on TTP and said biomarkers may comprise one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100, miR-151-3p, miR-592, miR-196b, miRmiR-338-3p, miR-133b, miR-15a, miR-552, miR-181a, miR-141, miR-545, miR-21*, miR-452, and miR-29c, miR-19b-1*, miR-664, miR-1285, miR-155, miR-532-3p, miR-1, miR-1227, miR-365, miR-145 and miR-625, miR-19b, miR-148a, miR-449a, miR-106b, miR-18b, miR-379, miR-193a-3p, miR-636, miR-1251, miR-449 b, miR-449, miR-455, miR-501.

Thus, for example if the one or more miRNAs in a sample is found to have an expression level which is close to the predetermined control level, wherein the predetermined control level is the average level in a patient with a similar disease where said other patients have been treated with an anti-angiogenic treatment and have a long time to disease progression (TTP), the sample has a high probability of being associated with an increase in TTP.

In one embodiment, said miRNA biomarkers may be used to distinguish between cancer patients, for whom anti-angiogenic treatment increases TTP vs. cancer patients for whom anti-angiogenic treatment has little or no effect on TTP and said biomarkers may comprise one or more miRNAs selected from the group consisting of miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

Thus, for example if the one or more miRNAs in a sample is found to have an expression level which is close to the predetermined control level, wherein the predetermined control level is the average level in a patient with a similar disease where said other patients have been treated with an anti-angiogenic treatment and have a long time to disease progression (TTP), the sample has a high probability of being associated with an increase in TTP.

In one embodiment, said miRNA biomarkers may be used to distinguish between cancer patients, for whom anti-angiogenic treatment increases TTP vs. cancer patients for whom anti-angiogenic treatment has little or no effect on TTP and said biomarkers may comprise one or more miRNAs selected from the group consisting of miR-17*, miR-22, miR-155, miR-185, miR-196b, miR-204, miR-214*, miR-382, miR-455, miR-545, miR-592 and miR-664.

Thus, for example if the one or more miRNAs in a sample is found to have an expression level which is close to the predetermined control level, wherein the predetermined control level is the average level in a patient with a similar disease where said other patients have been treated with an anti-angiogenic treatment and have a long time to disease progression (TTP), the sample has a high probability of being associated with an increase in TTP.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group of miR-382, miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p, and miR-29c.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group of miR-196b, miR-592, miR-185, miR-545, miR-29b, miR-204, miR-15a, miR-455-5p, miR-22, miR-338-3p, miR-19b, miR-143, miR-382, miR-660, miR-148a, miR-155, miR-449a, miR-106b, miR-141, miR-18b, miR-379, miR-214*, miR-552, miR-29c, miR-1227, miR-625, miR-181a, miR-193a-3p, miR-497, and miR-636.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group of miR-15a, miR-22, miR-29b, miR-148a, miR-155, miR-181a, miR-196b, miR-204, miR-214*, miR-338-3p, miR-382, miR-449a, miR-449 b, miR-455-5p, miR-497, miR-545, miR-552, and miR-592.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100, miR-151-3p, miR-592, miR-196b, miRmiR-338-3p, miR-133b, miR-15a, miR-552, miR-181a, miR-141, miR-545, miR-21*, miR-452, and miR-29c, miR-19b-1*, miR-664, miR-1285, miR-155, miR-532-3p, miR-1, miR-1227, miR-365, miR-145 and miR-625, miR-19b, miR-148a, miR-449a, miR-106b, miR-18b, miR-379, miR-193a-3p, miR-636, miR-1251, miR-449 b, miR-449, miR-455, miR-501.

Thus, for example ilf the one or more miRNAs in a sample is found to have an expression level which is close to the predetermined control level, wherein the predetermined control level is the average level in a patient with a similar disease where said other patients have been treated with an anti-angiogenic treatment and have a long time to death (OS), the sample has a high probability of being associated with an increase in OS.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group consisting of miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

Thus, for example if the one or more miRNAs in a sample is found to have an expression level which is close to the predetermined control level, wherein the predetermined control level is the average level in a patient with a similar disease where said other patients have been treated with an anti-angiogenic treatment and have a long time to death (OS), the sample has a high probability of being associated with an increase in OS.

In one embodiment, said miRNA biomarkers may be used to distinguish between cancer patients, for whom anti-angiogenic treatment increases OS vs. cancer patients for whom anti-angiogenic treatment has little or no effect on OS, and said biomarkers may comprise one or more miRNAs selected from the group consisting of miR-17*, miR-145, miR-155, miR-185, miR-196b, miR-204, miR-214*, miR-382, miR-449, miR-455, miR-501, miR-545, miR-592 and miR-664.

Thus, for example if the one or more miRNAs in a sample is found to have an expression level which is close to the predetermined control level, wherein the predetermined control level is the average level in a patient with a similar disease where said other patients have been treated with an anti-angiogenic treatment and have a long time to death (OS), the sample has a high probability of being associated with an increase in OS.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group consisting of miR-29b, miR-204, miR-214*, miR-382, and miR-497.

In one embodiment, said miRNA biomarkers may be used to distinguish between cancer patients, for whom anti-angiogenic treatment has good efficacy vs. cancer patients for whom anti-angiogenic treatment has little or no efficacy, and said biomarkers may comprise one or more miRNAs selected from the group consisting of miR-193b*, miR-145*, miR-29b, miR-22, miR-382, and miR-196b.

In one embodiment, said miRNA biomarkers may be used to distinguish between cancer patients, for whom chemotherapeutic treatment has good effect, for example for whom chemotherapeutic treatment increases TTP and/or OS vs. cancer patients for whom chemotherapeutic treatment has little or no effect, for example for whom chemotherapeutic treatment has little of no effect on TTP and/or OS. Said miRNA biomarkers may be any of the miRNA biomarkers described herein above.

The miRNA biomarkers as disclosed herein may in one embodiment be used (or measured; correlated) alone.

The miRNA biomarkers as disclosed herein may in another embodiment be used in combination, comprising at least two miRNA biomarkers.

It follows, that the combination of miRNA biomarkers as disclosed herein may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs, for example 20 miRNAs, as selected from the deregulated miRNA biomarkers disclosed herein.

The combination of miRNA biomarkers as disclosed may in another embodiment consist of less than 10 miRNAs, such as less than 9 miRNAs, for example less than 8 miRNAs, such as less than 7 miRNAs, for example less than 6 miRNAs, such as less than 5 miRNAs, for example less than 4 miRNAs, such as less than 3 miRNAs.

In one embodiment the present invention relates to a method of predicting the efficacy of an anti-angiogenic treatment in an individual suffering from cancer, wherein the primary tumor of said cancer is localized to sigmoid colon, rectum and/or rect-sigmoid colon.

In this embodiment the expression level of at least one miRNA is measured in a sample obtained from said individual. Said miRNA may be selected from the group consisting of miR-17*, miR-22, miR-145, miR-155, miR-185, miR-196b, miR-204, miR-214*, miR-382, miR-449, miR-455, miR-501, miR-545, miR-552, miR-592, and miR-664.

In one embodiment the expression level of a combination of miRNAs is determined, said combination comprising:

a) miR-664 and
b) one or more miRNAs selected from the group consisting of miR-17*, miR-22, miR-145, miR-155, miR-185, miR-196b, miR-204, miR-214*, miR-382, miR-449, miR-455, miR-501, miR-545, miR-552, and miR-592.

In one embodiment the expression level of a combination of miRNAs is determined, said combination comprising:

a) miR-664 and
b) miR-455 and
c) optionally one or more miRNAs selected from the group consisting of miR-17*, miR-22, miR-145, miR-155, miR-185, miR-196b, miR-204, miR-214*, miR-382, miR-449, miR-501, miR-545, miR-552, and miR-592.

In one embodiment the expression level of a combination of miRNAs is determined said combination comprising:

a) miR-664 and
b) one or more miRNAs selected from the group consisting of miR-17*, miR-185, miR-382, and miR-455.

### Classifier

Classifiers are relationships between sets of input variables, usually known as features, and discrete output variables, known as classes. Classes are often centred on the key questions of who, what, where and when. A classifier can intuitively be thought of as offering an opinion about whether, for instance, an individual associated with a given feature set is a member of a given class.

In other words, a classifier is a predictive model that attempts to describe one column (the label) in terms of others (the attributes). A classifier is constructed from data where the label is known, and may be later applied to predict label values for new data where the label is unknown. Internally, a classifier is an algorithm or mathematical formula that predicts one discrete value for each input row. For example, a classifier built from a dataset of iris flowers could predict the type of a presented iris given the length and width of its petals and stamen. Classifiers may also produce probability estimates for each value of the label. For example, a classifier built from a dataset of cars could predict the probability that a specific car was built in the United States.

### Sensitivity and Specificity

Sensitivity and specificity are statistical measures of the performance of a binary classification test. The sensitivity (also called recall rate in some fields) measures the proportion of actual positives which are correctly identified as such (i.e. the percentage of sick people who are identified as having the condition); and the specificity measures the proportion of negatives which are correctly identified (i.e. the percentage of well people who are identified as not having the condition). They are closely related to the concepts of type I and type II errors.

For any test, there is usually a trade-off between each measure. For example in a manufacturing setting in which one is testing for faults, one may be willing to risk discarding functioning components (low specificity), in order to increase the chance of identifying nearly all faulty components (high sensitivity). This trade-off can be represented graphically using a ROC curve.

$$sensitivity = \frac{number of True Positives}{number of True Positives + number of False Negatives}$$

A sensitivity of 100% means that the test recognizes all sick people as such. Thus in a high sensitivity test, a negative result is used to rule out the disease.

Sensitivity alone does not tell us how well the test predicts other classes (that is, about the negative cases). In the binary classification, as illustrated above, this is the corresponding specificity test, or equivalently, the sensitivity for the other classes. Sensitivity is not the same as the positive predictive value (ratio of true positives to combined true and false positives), which is as much a statement about the proportion of actual positives in the population being tested as it is about the test.

The calculation of sensitivity does not take into account indeterminate test results. If a test cannot be repeated, the options are to exclude indeterminate samples from analyses (but the number of exclusions should be stated when quoting sensitivity), or, alternatively, indeterminate samples can be treated as false negatives (which gives the worst-case value for sensitivity and may therefore underestimate it).

$$specificity = \frac{number of True Negatives}{number of True Negatives + number of False Positives}$$

A specificity of 100% means that the test recognizes all healthy people as healthy. Thus a positive result in a high specificity test is used to confirm the disease. The maximum is trivially achieved by a test that claims everybody healthy regardless of the true condition. Therefore, the specificity alone does not tell us how well the test recognizes positive cases. We also need to know the sensitivity of the test to the class, or equivalently, the specificities to the other classes. A test with a high specificity has a low Type I error rate.

Specificity is sometimes confused with the precision or the positive predictive value, both of which refer to the fraction of returned positives that are true positives. The distinction is critical when the classes are different sizes. A test with very high specificity can have very low precision if there are far more true negatives than true positives, and vice versa.

The accuracy of a measurement system is the degree of closeness of measurements of a quantity to its actual (true) value. The precision of a measurement system, also called reproducibility or repeatability, is the degree to which repeated measurements under unchanged conditions show the same results.

Accuracy is also used as a statistical measure of how well a binary classification test correctly identifies or excludes a condition. That is, the accuracy is the proportion of true results (both true positives and true negatives) in the population. It is a parameter of the test:

$$accuracy = \frac{number of true positives + number of true negatives}{\begin{matrix}numbers of true positives + false positives + \\ false negatives + true negatives\end{matrix}}$$

An accuracy of 100% means that the measured values are exactly the same as the given values.

On the other hand, precision is defined as the proportion of the true positives against all the positive results (both true positives and false positives)

$$precision = \frac{number of true positives}{number of true positives + false positives}$$

miRNA classifier of the present invention

The miRNA classifiers according to the present invention are the relationships between sets of input variables, i.e. the miRNA expression in a sample of an individual, and discrete output variables, i.e. distinction between e.g. predicted efficacy of an anti-angiogenic treatment of an individual suffering from cancer and/or predicted efficacy of a chemotherapeutic treatment. Thus, the classifier assigns a given sample to a given class with a given probability.

Distinction, differentiation or characterisation of a sample is used herein as being capable of predicting with a high sensitivity and specificity if a given sample of unknown diagnosis belongs to one of two classes (two-way classifier).

Platt's probabilistic outputs for Support Vector Machines (Platt, J. in Smola, A. J, et al. (eds.) Advances in large margin classifiers. Cambridge, 2000; incorporated herein by reference) is useful for applications that require posterior class probabilities. Also incorporated by reference herein is Platt J. Advances in Large Classifiers. Cambridge, Mass.: MIT Press, 1999.

In one embodiment, the p-value for preferred miRNAs to predict whether a sample belongs to the class of cancer patients, for whom anti-angiogenic treatment has good efficacy is a number falling in the range of from 0 to 0.05, preferably from 0 to 0.04, more preferably from 0 to 0.03, even more preferably from 0 to 0.02, yet more preferably from 0 to 0.01, even more preferably from 0 to 0.008.

Thus it is preferred that the p-value for preferred miRNAs to predict whether a sample belongs to the class of cancer patients, for whom anti-angiogenic treatment increases TTP is a number falling in the range of from 0 to 0.05, preferably from 0 to 0.04, more preferably from 0 to 0.03, even more preferably from 0 to 0.02, yet more preferably from 0 to 0.01, even more preferably from 0 to 0.008.

It is also preferred that the p-value for preferred miRNAs to predict whether a sample belongs to the class of cancer patients, for whom anti-angiogenic treatment has increases OS is a number falling in the range of from 0 to 0.05, preferably from 0 to 0.04, more preferably from 0 to 0.03, even more preferably from 0 to 0.02, yet more preferably from 0 to 0.01, even more preferably from 0 to 0.008.

The classifier according to the present invention may in one embodiment consist of 2 miRNAs, such as 3 miRNAs, for example 4 miRNAs, such as 5 miRNAs, for example 6 miRNAs, such as 7 miRNAs, for example 8 miRNAs, such as 9 miRNAs, for example 10 miRNAs, such as 11 miRNAs, for example 12 miRNAs, such as 13 miRNAs, for example 14 miRNAs, such as 15 miRNAs, for example 16 miRNAs, such as 17 miRNAs, for example 18 miRNAs, such as 19 miRNAs. Preferably the classifier consisting of 2 miRNAs, such as 3 miRNAs, for example 4 miRNAs, such as 5 miRNAs, for example 6 miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p, preferably from the group consisting of miR-193b*, miR-145*, miR-29b, miR-22, miR-382, and miR-196b, preferably from the group consisting of miR-17*, miR-22, miR-145, miR-155, miR-185, miR-196b, miR-204, miR-214*, miR-382, miR-449, miR-455, miR-501, miR-545, miR-552, miR-592 and miR-664.

Distinction, differentiation or characterisation of a sample is used herein as being capable of predicting with a high sensitivity and specificity if a given sample from a cancer patient with unknown status belongs to one of two classes (two-way classifier).

In one aspect, the present invention relates to a two-way miRNA classifier for characterising a sample obtained from an individual, wherein said miRNA classifier comprises or consists of one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p, preferably from the group consisting of miR-193b*, miR-145*, miR 29b, miR-22, miR-382 and miR-196b, preferably from the group consisting of miR-17*, miR-22, miR-145, miR-155, miR-185, miR-196b, miR-204, miR-214*, miR-382, miR-449, miR-455, miR-501, miR-545, miR-552, miR-592 and miR-664, wherein the classifier distinguishes cancer patients, wherein said distinction is given as a prediction probability for said sample of belonging to either class, said probability being a number falling in the range of from 0 to 1.

In another aspect, the present invention relates to a two-way miRNA classifier for characterising a sample obtained from an individual, wherein said miRNA classifier comprises or consists of one or more miRNAs selected from the group consisting of miR-382, miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p, preferably from the group consisting of miR-193b*, miR-145*, miR-29b, miR-22, miR-382 and miR-196b, preferably from the group consisting of miR-17*, miR-22, miR-145, miR-155, miR-185, miR-196b, miR-204, miR-214*, miR-382, miR-449, miR-455, miR-501, miR-545, miR-552, miR-592 and miR-664, and distinguishes cancer patients, for whom anti-angiogenic treatment has good efficacy from cancer patients for whom anti-angiogenic treatment has little or no efficacy, wherein said distinction is given as a prediction probability for said sample of belonging to either class, said probability being a number falling in the range of from 0 to 1.

In one embodiment, the two-way miRNA classifier further comprises one or more additional miRNAs selected from the deregulated miRNA biomarkers as disclosed herein above.

In one embodiment, the two-way miRNA classifiers further comprises one or more additional miRNAs, such as 1 additional miRNA, for example 2 additional miRNAs, such as 3 additional miRNA, for example 4 additional miRNAs, such as 5 additional miRNA, for example 6 additional miRNAs, such as 7 additional miRNA, for example 8 additional miRNAs, such as 9 additional miRNA, for example 10 additional miRNAs, such as 11 additional miRNA, for example 12 additional miRNAs, such as 13 additional miRNA, for example 14 additional miRNAs, such as 15 additional miRNAs, for example 16 additional miRNAs, such as 17 additional miRNA, for example 18 additional miRNAs, such as 19 additional miRNAs, for example 20 additional miRNAs selected from the miRNA biomarkers as disclosed herein above, e.g. from the miRNAs disclosed in Tables 1, 2, 3 and 4, preferably from the miRNAs disclosed in Tables 5 and 6.

In one embodiment, the miRNAs to be used with the present invention may be a two-way miRNA classifier for characterising a sample obtained from an individual, wherein said miRNA classifier comprises or consists of at least one, preferably at least two, more preferably at least three miRNAs selected from the group consisting of miR-193b*, miR-145*, miR-29b, miR-22, miR-382, miR-196b, combined with at least one miRNAs selected from the group consisting of miR-370, miR-497, miR-29c*, miR-501-5p, miR-146 b-3p, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p combined with at least one, preferably at least two, more preferably at least three miRNAs selected from the group consisting of miRNAs disclosed in Tables 1, 2, 3 and 4, preferably from the miRNAs disclosed in Tables 5 and 6, wherein the miRNA classifier distinguishes cancer patients, for whom anti-angiogenic treatment has good efficacy vs. cancer patients for whom anti-angiogenic treatment has little or no efficacy.

In one embodiment, the miRNAs to be used with the present invention may be a two-way miRNA classifier for characterising a sample obtained from an individual, wherein said miRNA classifier comprises or consists of at least one, preferably at least two, more preferably at least three miRNAs selected from the group consisting of miR-193b*, miR-145*, miR-29b, miR-22, miR-382 and miR-196b combined with at least one, preferably at least two, more preferably at least three miRNAs selected from the group consisting of miR-370, miR-497, miR-29c*, miR-501-5p, miR-146 b-3p, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p, wherein the miRNA classifier distinguishes cancer patients, for whom anti-angiogenic treatment has good efficacy vs. cancer patients for whom anti-angiogenic treatment has little or no efficacy.

In one embodiment, the miRNAs to be used with the present invention may be a two-way miRNA classifier for characterising a sample obtained from an individual, wherein said miRNA classifier comprises or consists of at least one, preferably at least two, more preferably at least three miRNAs selected from the group consisting of miR-17*, miR-22, miR-145, miR-155, miR-185, miR-196b, miR-204, miR-214*, miR-382, miR-449, miR-455, miR-501, miR-545, miR-552, miR-592 and miR-664 combined with at least one, preferably at least two, more preferably at least three miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664 wherein the miRNA classifier distinguishes cancer patients, for whom anti-angiogenic treatment has good efficacy vs. cancer patients for whom anti-angiogenic treatment has little or no efficacy.

The miRNA classifiers disclosed herein in a particular embodiment has a sensitivity of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has an accuracy of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a specificity of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a negative predictive value for malignancies of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value for malignancies of at least 80%, such as at least 81%, for example at least 82%, such as at least 83%, for example at least 84%, such as at least 85%, for example at least 86%, such as at least 87%, for example at least 88%, such as at least 89%, for example at least 90%, such as at least 91%, for example at least 92%, such as at least 93%, for example at least 94%, such as at least 95%.

The miRNA classifiers disclosed herein in a particular embodiment has a positive predictive value or a negative predictive value for efficacy of anti-angiogenic treatment of between 80-85%, such as 85-90%, for example 90-95%, such as 95-96%, for example 96-97%, such as 97-98%, for example 98-99%, such as 99-100%.

Methods for Diagnosis Employing the miRNA Classifier and/or Biomarkers of the Present Invention

The invention in one aspect relates to a method for predicting the efficacy of an anti-angiogenic treatment in an individual suffering from cancer, comprising measuring the expression level of at least one miRNA in a sample obtained from said individual, wherein said miRNA may be any of the miRNAs described herein above in the sections “miRNA biomarkers of the present invention” and “miRNA classifier of the present invention” and/or measuring the expression level of at least one miRNA classifier, which may be any of the miRNA classifiers described herein above in the section “miRNA classifier of the present invention”.

In a preferred embodiment the expression level of at least one miRNA in a sample obtained from an individual is determined, wherein said miRNA is selected from the group consisting of miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

It is understood that said difference in miRNA expression level in a preferred embodiment is a relative difference between said miRNA's expression levels. Accordingly both an increase and a decrease in expression may be relevant and depends on the particular miRNA examined.

Thus, in relation to miR-664, if a sample has a high expression level, then the sample has a high probability of being associated with a good efficacy of anti-angiogenic treatment. A high expression level is preferably an expression level in the highest 3 quartiles, more preferably in the highest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-664 by QPCR, then the Ct value is preferably in the lowest 3 quartiles, more preferably in the lowest 2 quartiles, when compared to the Ct-value of miR-664 determined in at least 50, such as at least 100 individuals suffering from colorectal cancer.

Thus, in relation to miR-17*, if a sample has a high expression level, then the sample has a high probability of being associated with a good efficacy of anti-angiogenic treatment. A high expression level is preferably an expression level in the highest 3 quartiles, more preferably in the highest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-17* by QPCR, then the Ct value is preferably in the lowest 3 quartiles, more preferably in the lowest 2 quartiles, when compared to the Ct-value of miR-17* determined in at least 50, such as at least 100 individuals suffering from colorectal cancer.

Thus, in relation to miR-196b, if a sample has a high expression level, then the sample has a high probability of being associated with a good efficacy of anti-angiogenic treatment. A high expression level is preferably an expression level in the highest 3 quartiles, more preferably in the highest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-196b by QPCR, then the Ct value is preferably in the lowest 3 quartiles, more preferably in the lowest 2 quartiles, when compared to the Ct-value of miR-196b determined in at least 50, such as at least 100 individuals suffering from colorectal cancer.

Thus, in relation to miR-552, if a sample has a high expression level, then the sample has a high probability of being associated with a good efficacy of anti-angiogenic treatment. A high expression level is preferably an expression level in the highest 3 quartiles, more preferably in the highest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-552 by QPCR, then the Ct value is preferably in the lowest 3 quartiles, more preferably in the lowest 2 quartiles, when compared to the Ct-value of miR-552 determined in at least 50, such as at least 100 individuals suffering from colorectal cancer.

Thus, in relation to miR-592, if a sample has a high expression level, then the sample has a high probability of being associated with a good efficacy of anti-angiogenic treatment. A high expression level is preferably an expression level in the highest 3 quartiles, more preferably in the highest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-592 by QPCR, then the Ct value is preferably in the lowest 3 quartiles, more preferably in the lowest 2 quartiles, when compared to the Ct-value of miR-592 determined in at least 50, such as at least 100 individuals suffering from colorectal cancer.

Thus, in relation to miR-22, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-22 by QPCR, then the Ct value is preferably in the highest 3 quartiles, more preferably in the highest 2 quartiles, when compared to the Ct-value of miR-22 determined in at least 50, such as at least 100 individuals suffering from colorectal cancer.

Thus, in relation to miR-145, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-145 by QPCR, then the Ct value is preferably in the highest 3 quartiles, more preferably in the highest 2 quartiles, when compared to the Ct-value of miR-145 determined in at least 50, such as at least 100 individuals suffering from colorectal cancer.

Thus, in relation to miR-155, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-155 by QPCR, then the Ct value is preferably in the highest 3 quartiles, more preferably in the highest 2 quartiles, when compared to the Ct-value of miR-155 determined in at least 50, such as at least 100 individuals suffering from colorectal cancer.

Thus, in relation to miR-185, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-185 by QPCR, then the Ct value is preferably in the highest 3 quartiles, more preferably in the highest 2 quartiles, when compared to the Ct-value of miR-185 determined in at least 50, such as at least 100 individuals suffering from colorectal cancer.

Thus, in relation to miR-214, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-214 by QPCR, then the Ct value is preferably in the highest 3 quartiles, more preferably in the highest 2 quartiles, when compared to the Ct-value of miR-214 determined in at least 50, such as at least 100 individuals suffering from colorectal cancer.

Thus, in relation to miR-382, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-382 by QPCR, then the Ct value is preferably in the highest 3 quartiles, more preferably in the highest 2 quartiles, when compared to the Ct-value of miR-382 determined in at least 50, such as at least 100 individuals suffering from colorectal cancer.

Thus, in relation to miR-449, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-449 by QPCR, then the Ct value is preferably in the highest 3 quartiles, more preferably in the highest 2 quartiles, when compared to the Ct-value of miR-449 determined in at least 50, such as at least 100 individuals suffering from colorectal cancer.

Thus, in relation to miR-455, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer.

The expression level may be determined as described herein below in the section “Sample analysis”. In embodiments of the invention wherein the expression level is determined by determining the Ct value of miR-455 by QPCR, then the Ct value is preferably in the highest 3 quartiles, more preferably in the highest 2 quartiles, when compared to the Ct-value of miR-455 determined in at least 50, such as at least 100 individuals suffering from colorectal cancer.

Thus, in relation to miR-501, if a sample has a low expression level, then the sample has a high probability of being associated with a good efficacy of anti-angiogenic treatment. A low expression level is preferably an expression level in the lowest 3 quartiles, more preferably in the lowest 2 quartiles, when compared to the expression level of at least 50, such as at least 100 individuals suffering from colorectal cancer. The expression level may be determined as described herein below in the section “Sample analysis”. In embodiments of the invention wherein the expression level is determined by determining the Ct value of miR-501 by QPCR, then the Ct value is preferably in the highest 3 quartiles, more preferably in the highest 2 quartiles, when compared to the Ct-value of miR-501 determined in at least 50, such as at least 100 individuals suffering from colorectal cancer.

In one embodiment, said method further comprises the step of extracting RNA from a sample collected from an individual, by any means as disclosed herein elsewhere.

In one embodiment, said method further comprises the step of correlating the miRNA expression level of at least one of said miRNAs to a predetermined control level. The predetermined control level may in one embodiment be the level of miRNA expression in patients with known good efficacy of an anti-angiogenic treatment.

In another embodiment, the predetermined control level is the average level in patients with similar disease where said other patient have been treated with an anti-angiogenic treatment and have shown a long time to disease progression (TTP).

In yet another embodiment, the predetermined control level is the average level in patients with similar disease where said other patient have been treated with an anti-angiogenic treatment and have shown a long time to death (OS).

In one embodiment, said method further comprises the step of determining if said individual is suffering from cancer, preferably to determine if said individual is suffering from colorectal cancer.

In one embodiment, said method further comprises the step of obtaining a sample from an individual suffering from cancer, preferably a sample comprising cancer cells from said individual, by any means as disclosed herein elsewhere.

Said sample is in one particular embodiment a tissue sample comprising part of the primary tumour or a metastasis from said individual. Thus in embodiments of the invention, where the individual is suffering from colorectal cancer, the sample may preferably be a sample comprising at least part of the primary colorectal tumor.

In one embodiment, said miRNA expression level is altered as compared to the expression level in a control sample. Said control sample may in one embodiment be normal tissue.

In one embodiment, said cancer is a colorectal cancer, for example a cancer selected from cancers of the colon, cancers of the rectum and cancers of the appendix.

In a preferred embodiment of the present invention, said cancer is a colorectal cancer selected from cancers of the sigmoid colon, cancers of the rectum and cancers of the recto-sigmoid colon.

In one embodiment the expression levels of said miRNAs, such as any of the miRNAs described herein above in the sections “miRNA biomarkers of the present invention” and “miRNA classifier of the present invention” and/or measuring the expression level of at least one miRNA classifier, are measured by qPCR and the difference in expression is calculated.

Thus, the Ct-value of the specific miRNA in a predetermined control sample may be determined by QPCR. The Ct-value as used herein is the number of amplification cycles required for the fluorescent signal to cross the background level. The Ct-value of said miRNA in a sample from an individual suffering from cancer may then be measured by QPCR. A difference in Ct-values corresponds to a difference in expression level of said miRNA. Preferably said difference in Ct-values is at least 2, such as least 3, for example at least 4, such as at least 5. The Ct value may for example be determined as described herein below in the section “RT-QPCR”.

By way of example, if the predetermined control level of a specific miRNA corresponds to a Ct-values of 25 as determined by QPCR, then a high expression level is a Ct-value of less than 25, whereas a low expression of said miRNA is a Ct-value higher than 25.

When determining expression level of a given miRNA as described herein above in the section “miRNA biomarkers of the present invention”, then this may for example be done by determining the Ct value of said miRNA by QPCR in a sample comprising cancer cells from an individual suffering from cancer and comparing the Ct value to a predetermined control Ct value of said miRNA. The predetermined control Ct value is preferably the Ct value of said miRNA found by QPCR in patients with known good efficacy of an anti-angiogneic treatment, for example the average Ct value of said miRNA found by QPCR in at least 5, such as at least 10, for example at least 15, such as at least 20 patients with known good efficacy of an anti-angiogneic treatment. Preferably, the Ct value in said sample is determined in the same manner as the predetermined control Ct value using the same settings and materials for the QPCR. In general, when the difference in Ct-values between the Ct value of said sample and the predetermined control Ct value is less than 5, preferably less than 4, more preferably less than 3, such as less than 2, then the expression level of said miRNA is considered “close” to the predetermined control level.

In one embodiment the expression levels of said miRNAs, such as any of the miRNAs described herein above in the sections “miRNA biomarkers of the present invention” and “miRNA classifier of the present invention” and/or measuring the expression level of at least one miRNA classifier, are measured by in situ hybridization in tissue samples and the difference in expression is calculated.

In one embodiment hazard ratios are used to evaluate whether there is a difference between the specific miRNAs in a sample from a patient suffering from cancer and the same specific miRNAs in a group of control patients. Hazard ratio (HR) is a known statistical measurement. A HR of one means equivalence in the hazard rate in the two groups, whereas a HR other than one indicates a difference in hazard rate between the groups.

A preferred miRNA of the present invention has a HR differing from one. The larger the difference is from one, the better a biomarker or classifier the specific miRNA is.

In one embodiment of the present invention the miRNA used as a biomarker or classifier is any one of the miRNAs found herein below in table 1, 2, 3, 4, 5, 6, 9 and 11 with a HR different from one.

In a preferred embodiment, the miRNA used as a biomarker or classifier is any one of the miRNAs found in table 11 with a HR different from one.

In a preferred embodiment of the present invention the miRNA used as a biomarker or classifier is any miRNA with a HR below 0.8 or above 1.1. Very preferred miRNA used as a biomarker or a classifier is any miRNA with a HR below 0.5 or above 1.2.

In a further embodiment of the present invention, any of the above-mentioned methods may be is used in combination with at least one additional method for predicting the efficacy of an anti-agiogenic treatment.

Said at least one additional diagnostic method may in one embodiment be selected from the group consisting of CT (X-ray computed tomography) scanning, MRI (magnetic resonance imaging), scintillation counting, blood sample analysis, ultrasound imaging, cytology, histology and assessment of risk factors. These are described herein above.

A model for predicting the efficacy of an anti-angiogenic treatment in an individual suffering from cancer by employing the miRNA classifier of the present invention.

In one aspect, the present invention relates to a model for predicting the efficacy of an anti-angiogenic treatment in an individual suffering from cancer, comprising
i) providing a set of input data to the miRNA classifier according to the present invention, and
ii) determining the predicted efficacy of an anti-angiogenic treatment of said individual.

In one embodiment, said input data comprises or consists of the miRNA expression profile of one or more of miRNAs selected from the group of miRNAs mentioned in Tables 1, 2, 3 and 4, preferably from the group of miRNAs mentioned in Tables 5 and 6.

In another embodiment, said input data comprises or consists of the miRNA expression profile of one or more of miRNA selected from the group of miRNAs mentioned in Table 1 and one or more of miRNA selected from the group of miRs mentioned in Table 2, wherein the miRNAs selected from miRNAs mentioned in Table 1 is different from the miRNAs selected from miRs of Table 2.

In another embodiment, said input data comprises or consists of the miRNA expression profile of one or more of miRNAs selected from the group of miRNAs mentioned in Table 3 and one or more of miRNAs selected from the group of miRNAs mentioned in Table 4, wherein the miRNAs selected from miRNAs mentioned in Table 3 is different from the miRNAs selected from miRNAs of Table 4.

In another embodiment, said input data comprises or consists of the miRNA expression profile of one or more of miRNAs selected from the group of miRNAs mentioned in Table 5 and one or more of miRNAs selected from the group of miRNAs mentioned in Table 6, wherein the miRNAs selected from miRNAs mentioned in Table 5 is different from the miRNAs selected from miRNAs of Table 6.

In another embodiment, said input data comprises or consists of the miRNA expression profile of one or more of miR-1, miR-17*, miR-22, miR-29a*, miR-29b, miR-145*, miR-185, miR-193b*, miR-204, miR-214*, miR-365, miR-382, miR-497, miR-501-5p, miR-664, miR-1251, miR-15a, miR-29b, miR-148a, miR-155, miR-181a, miR-196b, miR-338-3p, miR-449a, miR-449 b, miR-455-5p, miR-545, miR-552, and miR-592.

In yet another embodiment said input data comprises or consists of the miRNA expression profile of one or more of miR selected from the group consisting of miR-1, miR-17*, miR-22, miR-29a*, miR-29b, miR-145*, miR-185, miR-193b*, miR-204, miR-214*, miR-365, miR-382, miR-497, miR-501-5p, miR-664, and miR-1251 and one or more of miR selected from the group consisting of miR-15a, miR-148a, miR-155, miR-181a, miR-196b, miR-338-3p, miR-382, miR-449a, miR-449 b, miR-455-5p, miR-545, miR-552, and miR-592, wherein the input data comprises the expression profile of at least two different miRNAs.

In another embodiment, said input data comprises or consists of the miRNA expression profile of one or more of miR-29b, miR-204, miR-214*, miR-382, and miR-497.

In another embodiment, said input data comprises or consists of miRNA expression profile of one or more of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

In another embodiment, said input data comprises or consists of miRNA expression profile of one or more of miR-17*, miR-22, miR-145, miR-155, miR-185, miR-196b, miR-204, miR-214*, miR-382, miR-449, miR-455, miR-501, miR-545, miR-552, miR-592, and miR-664.

### Sample Type

The sample according to the present invention is extracted from an individual and used for miRNA profiling for the subsequent prediction of the efficacy of an anti-angiogenic treatment.

The sample may be collected from an individual or a cell culture, preferably an individual. The individual may be any animal, such as a mammal, including human beings. In a preferred embodiment, the individual is a human being.

The individual is an individual suffering from cancer and the sample preferably comprises cancer cells. Thus, preferably said sample comprises at least part of the primary tumor or at least part of a metastasis. Preferably the individual is suffering from colorectal cancer, and more preferably the individual is suffering from colon cancer, where the primary tumour is located to the sigmoid colon, the rectum and/or the recto-sigmoid colon.

In a particular embodiment, the sample is taken from the colon, the rectum or the appendix of a human being. In a preferred embodiment, the sample is taken from the sigmoid colon, the rectum and/or the recto-sigmoid colon. This is in particular the case in embodiments of the invention, where the individual is suffering from colorectal cancer, where the primary tumour is located to the sigmoid colon, the rectum and/or the recto-sigmoid colon. In such an instance, the sample may be denoted a tissue or blood sample.

The tissue sample may further comprises cells of the desmoplastic stroma surrounding the tumor, e.g. fibroblasts, inflammatory cells (e.g. macrophages and neutrofils) and endothelial cells.

### Sample Collection

In one embodiment, the sample is collected from the colorectal system of an individual by any available means, such as by fine-needle aspiration (FNA) using a needle with a maximum diameter of 1 mm; by core needle aspiration using a needle with a maximum diameter of above 1 mm (also called coarse needle aspiration or biopsy, large needle aspiration or large core aspiration); by biopsy; by cutting biopsy; by open biopsy; a surgical sample; or by any other means known to the person skilled in the art. In another embodiment, the sample is collected from an in vitro cell culture or a blood sample.

In a particular embodiment, the sample is a fine-needle aspirate from an individual. The fine-needle aspiration may be performed using a needle with a diameter of between 0.2 to 1.0 mm, such as 0.2 to 0.3 mm, for example 0.3 to 0.4 mm, such as 0.4 to 0.5 mm, for example 0.5 to 0.6 mm, such as 0.6 to 0.7 mm, for example 0.7 to 0.8 mm, such as 0.8 to 0.9 mm, for example 0.9 to 1.0 mm in diameter.

Said fine-needle aspiration may in one embodiment be a single fine-needle aspiration, or may in another embodiment comprise multiple fine-needle aspirations.

The diameter of the needle is indicated by the needle gauge. Various needle lengths are available for any given gauge. Needles in common medical use range from 7 gauge (the largest) to 33 (the smallest) on the Stubs scale. Although reusable needles remain useful for some scientific applications, disposable needles are far more common in medicine. Disposable needles are embedded in a plastic or aluminium hub that attaches to the syringe barrel by means of a press-fit (Luer) or twist-on (Luer-lock) fitting.

The fine-needle aspiration is in one embodiment performed using a needle gauge of between 20 to 33, such as needle gauge 20, for example needle gauge 21, such as needle gauge 22, for example needle gauge 23, such as needle gauge 24, for example needle gauge 25, such as needle gauge 26, for example needle gauge 27, such as needle gauge 28, for example needle gauge 29, such as needle gauge 30, for example needle gauge 31, such as needle gauge 32, for example needle gauge 33.

The fine-needle aspiration may in one embodiment be assisted, such as ultra-sound (US) guided fine-needle aspiration, x-ray guided fine-needle aspiration, endoscopic ultra-sound (EUS) guided fine-needle aspiration, Endobronchial ultrasound-guided fine-needle aspiration (EBUS), ultrasonographically guided fine-needle aspiration, stereotactically guided fine-needle aspiration, computed tomography (CT)-guided percutaneous fine-needle aspiration and palpation guided fine-needle aspiration.

The skin above the area to be biopsied may in one embodiment be swiped with an antiseptic solution and/or may be draped with sterile surgical towels. The skin, underlying fat, and muscle may in one embodiment be numbed with a local anesthetic. After the needle is placed into the mass, cells may be withdrawn by aspiration with a syringe.

In another embodiment, the sample is a blood sample extracted or drawn from an individual by any conventional method known to the skilled person. The blood may be drawn from a vein or an artery of an individual.

The sample extracted from an individual by any means as disclosed above may be transferred to a tube or container prior to analysis. The container may be empty, or may comprise a collection media of sorts.

The sample extracted from an individual by any means as disclosed above may be analysed essentially immediately, or it may be stored prior to analysis for a variable period of time and at various temperature ranges.

In one embodiment, the sample is stored at a temperature of between −200° C. to 37° C., such as between −200° to −100° C., for example −100° to −50° C., such as −50° to −25° C., for example 25° to −10° C., such as −10° to 0° C., for example 0° to 10° C., such as 10° to 20° C., for example 20° to 30° C., such as 30° to 37° C. prior to analysis.

In one embodiment, the sample is stored at −20° C. and/or −80° C.

In another embodiment, the sample is stored for between 15 minutes and 100 years prior to analysis, such as between 15 minutes and 1 hour, for example 1 to 2 hours, such as 2 to 5 hours, for example 5 to 10 hours, such as 10 to 24 hours, for example 24 hours to 48 hours, such as 48 to 72 hours, for example 72 to 96 hours, such as 4 to 7 days, such as 1 week to 2 weeks, such as 2 to 4 weeks, such as 4 weeks to 1 month, such as 1 month to 2 months, for example 2 to 3 months, such as 3 to 4 months, for example 4 to 5 months, such as 5 to 6 months, for example 6 to 7 months, such as 7 to 8 months, for example 8 to 9 months, such as 9 to 10 months, for example 10 to 11 months, such as 11 to 12 months, for example 1 year to 2 years, such as 2 to 3 years, for example 3 to 4 years, such as 4 to 5 years, for example 5 to 6 years, such as 6 to 7 years, for example 7 to 8 years, such as 8 to 9 years, for example 9 to 10 years, such as 10 to 20 years, for example 20 to 30 years, such as 30 to 40 years, for example 40 to 50 years, such as 50 to 75 years, for example 75 to 100 years prior to analysis.

In one embodiment, the sample is stored for a few days.

### Collection Media for Sample

A collection media according to the present invention is any media suitable for preserving and/or collecting a sample for immediate or later analysis.

In one embodiment, said collection media is a solution suitable for sample preservation and/or later retrieval of RNA (such as miRNA) from said sample.

In one embodiment, the collection media is an RNA preservation solution or reagent suitable for containing samples without the immediate need for cooling or freezing the sample, while maintaining RNA integrity prior to extraction of RNA (such as miRNA) from the sample. An RNA preservation solution or reagent may also be known as RNA stabilization solution or reagent or RNA recovery media, and may be used interchangeably herein. The RNA preservation solution may penetrate the harvested cells of the collected sample to retard RNA degradation to a rate dependent on the storage temperature.

The RNA preservation solution may be any commercially available solutions or it may be a solution prepared according to available protocols.

The commercially available RNA preservation solutions may for example be selected from RNAlater® (Ambion and Qiagen), PreservCyt medium (Cytyc Corp), PrepProtect™ Stabilisation Buffer (Miltenyi Biotec), Allprotect Tissue Reagent (Qiagen) and RNAprotect Cell Reagent (Qiagen). Protocols for preparing a RNA stabilizing solution may be retrieved from the internet (e.g. L. A. Clarke and M. D. Amaral: ‘Protocol for RNase-retarding solution for cell samples’, provided through The European Workin Group on CFTR Expression), or may be produced and/or optimized according to techniques known to the skilled person.

In another embodiment, the collection media will penetrate and lyse the cells of the sample immediately, including reagents and methods for isolating RNA (such as miRNA) from a sample that may or may not include the use of a spin column.

Said reagents and methods for isolating RNA (such as miRNA) is described herein below in the section ‘analysis of sample’.

Other collection media according to the present invention comprises any media such as water, sterile water, denatured water, saline solutions, buffers, PBS, TBS, Allprotect Tissue Reagent (Qiagen), cell culture media such as RPMI-1640, DMEM (Dulbecco's Modified Eagle Medium), MEM (Minimal Essential Medium), IMDM (Iscove's Modified Dulbecco's Medium), BGjB (Fitton-Jackson modification), BME (Basal Medium Eagle), Brinster's BMOC-3 Medium, CMRL Medium, CO₂-Independent Medium, F-10 and F-12 Nutrient Mixture, GMEM (Glasgow Minimum Essential Medium), IMEM (Improved Minimum Essential Medium), Leibovitz's L-15 Medium, McCoy's 5A Medium, MCDB 131 Medium, Medium 199, Opti-MEM, Waymouth's MB 752/1, Williams' Media E, Tyrode's solution, Belyakov's solution, Hanks' solution and other cell culture media known to the skilled person, tissue preservation media such as HypoThermosol®, CryoStor™ and Steinhardt's medium and other tissue preservation media known to the skilled person.

In one preferred embodiment, said collection media is means for fixation (preservation) of said tissue sample; a tissue fixative, such as formalin (formaldehyde) or the like.

Types of tissue fixation includes heat fixation, chemical fixation (Crosslinking fixatives—Aldehydes; Precipitating fixatives—Alcohols; Oxidising agents; Mercurials; Picrates; HOPE (Hepes-glutamic acid buffer-mediated organic solvent protection effect) Fixative), and Frozen Sections.

In one embodiment, the fixation time may be between 1 to 7 calendar days; such as 1 day, 2 days, 3 days, 4 days, 5 days, 6 days or 7 days.

It follows that the invention may be carried out on formalin fixed paraffin embedded tissue blocks (FFPE).

### Sample Analysis

After the sample is collected, it is subjected to analysis. In one embodiment, the sample is initially used for isolating or extracting RNA according to any conventional methods known in the art; followed by an analysis of the miRNA expression in said sample.

### Extraction of RNA

The RNA isolated from the sample may be total RNA, mRNA, microRNA, tRNA, rRNA or any type of RNA.

Conventional methods and reagents for isolating RNA from a sample comprise High Pure miRNA Isolation Kit (Roche), Trizol (Invitrogen), Guanidinium thiocyanate-phenol-chloroform extraction, PureLink™ miRNA isolation kit (Invitrogen), PureLink Micro-to-Midi Total RNA Purification System (invitrogen), RNeasy kit (Qiagen), miRNeasy kit (Qiagen), Oligotex kit (Qiagen), phenol extraction, phenol-chloroform extraction, TCA/acetone precipitation, ethanol precipitation, Column purification, Silica gel membrane purification, PureYield™ RNA Midiprep (Promega), PolyATtract System 1000 (Promega), Maxwell® 16 System (Promega), SV Total RNA Isolation (Promega), geneMAG-RNA/DNA kit (Chemicell), TRI Reagent@ (Ambion), RNAqueous Kit (Ambion), ToTALLY RNA™ Kit (Ambion), Poly(A)Purist™ Kit (Ambion) and any other methods, commercially available or not, known to the skilled person.

The RNA may be further amplified, cleaned-up, concentrated, DNase treated, quantified or otherwise analysed or examined such as by agarose gel electrophoresis, absorbance spectrometry or Bioanalyser analysis (Agilent) or subjected to any other post-extraction method known to the skilled person.

Methods for extracting and analysing an RNA sample are disclosed in Molecular Cloning, A Laboratory Manual (Sambrook and Russell (ed.), 3^{rd} edition (2001), Cold Spring Harbor Laboratory Press, Cold Spring Harbor, N.Y., USA.

### Microarray Analysis

The isolated RNA may be analysed by microarray analysis. In one embodiment, the expression level of one or more miRNAs is determined by the microarray technique.

A microarray is a multiplex technology that consists of an arrayed series of thousands of microscopic spots of DNA oligonucleotides or antisense miRNA probes, called features, each containing picomoles of a specific oligonucleotide sequence. This can be a short section of a gene or other DNA or RNA element that are used as probes to hybridize a DNA or RNA sample (called target) under high-stringency conditions. Probe-target hybridization is usually detected and quantified by fluorescence-based detection of fluorophore-labeled targets to determine relative abundance of nucleic acid sequences in the target. In standard microarrays, the probes are attached to a solid surface by a covalent bond to a chemical matrix (via epoxy-silane, amino-silane, lysine, polyacrylamide or others). The solid surface can be glass or a silicon chip, in which case they are commonly known as gene chip. DNA arrays are so named because they either measure DNA or use DNA as part of its detection system. The DNA probe may however be a modified DNA structure such as LNA (locked nucleic acid).

In one embodiment, the microarray analysis is used to detect microRNA, known as microRNA or miRNA expression profiling.

The microarray for detection of microRNA may be a microarray platform, wherein the probes of the microarray may be comprised of antisense miRNAs or DNA oligonucleotides. In the first case, the target is a labelled sense miRNA sequence, and in the latter case the miRNA has been reverse transcribed into cDNA and labelled.

The microarray for detection of microRNA may be a commercially available array platform, such as NCode™ miRNA Microarray Expression Profiling (Invitrogen), miRCURY LNA™ microRNA Arrays (Exiqon), microRNA Array (Agilent), μParaflo® Microfluidic Biochip Technology (LC Sciences), MicroRNA Profiling Panels (Illumina), Geniom® Biochips (Febit Inc.), microRNA Array (Oxford Gene Technology), Custom AdmiRNA™ profiling service (Applied Biological Materials Inc.), microRNA Array (Dharmacon-Thermo Scientific), LDA TaqMan analyses (Applied Biosystems), Taqman microRNA Array (Applied Biosystems) or any other commercially available array.

Microarray analysis may comprise all or a subset of the steps of RNA isolation, RNA amplification, reverse transcription, target labelling, hybridisation onto a microarray chip, image analysis and normalisation, and subsequent data analysis; each of these steps may be performed according to a manufacturers protocol.

It follows, that any of the methods as disclosed herein above e.g. for diagnosing of an individual may further comprise one or more of the steps of:
i) isolating miRNA from a sample,
ii) labelling of said miRNA,
iii) hybridising said labelled miRNA to a microarray comprising miRNA-specific probes to provide a hybridisation profile for the sample,
iv) performing data analysis to obtain a measure of the miRNA expression profile of said sample.

In another embodiment, the microarray for detection of microRNA is custom made.

A probe or hybridization probe is a fragment of DNA or RNA of variable length, which is used to detect in DNA or RNA samples the presence of nucleotide sequences (the target) that are complementary to the sequence in the probe. One example is a sense miRNA sequence in a sample (target) and an antisense miRNA probe. The probe thereby hybridizes to single-stranded nucleic acid (DNA or RNA) whose base sequence allows probe-target base pairing due to complementarity between the probe and target.

To detect hybridization of the probe to its target sequence, the probe or the sample is tagged (or labeled) with a molecular marker. Detection of sequences with moderate or high similarity depends on how stringent the hybridization conditions were applied—high stringency, such as high hybridization temperature and low salt in hybridization buffers, permits only hybridization between nucleic acid sequences that are highly similar, whereas low stringency, such as lower temperature and high salt, allows hybridization when the sequences are less similar. Hybridization probes used in microarrays refer to nucleotide sequences covalently attached to an inert surface, such as coated glass slides, and to which a mobile target is hybridized. Depending on the method the probe may be synthesised via phosphoramidite technology or generated by PCR amplification or cloning (older methods). To design probe sequences, a probe design algorithm may be used to ensure maximum specificity (discerning closely related targets), sensitivity (maximum hybridisation intensities) and normalised melting temperatures for uniform hybridisation.

### Rt-Qpcr

The isolated RNA may be analysed by quantitative (‘real-time’) PCR (QPCR). In one embodiment, the expression level of one or more miRNAs is determined by the quantitative polymerase chain reaction (QPCR or qPCR) technique.

Real-time polymerase chain reaction, also called quantitative polymerase chain reaction (Q-PCR/qPCR/RT-QPCR) or kinetic polymerase chain reaction, is a technique based on the polymerase chain reaction, which is used to amplify and simultaneously quantify a targeted DNA molecule. It enables both detection and quantification (as absolute number of copies or relative amount when normalized to DNA input or additional normalizing genes) of a specific sequence in a DNA sample.

The procedure follows the general principle of polymerase chain reaction; its key feature is that the amplified DNA is quantified as it accumulates in the reaction in real time after each amplification cycle. Two common methods of quantification are the use of fluorescent dyes that intercalate with double-stranded DNA, and modified DNA oligonucleotide probes that fluoresce when hybridized with a complementary DNA. Frequently, real-time polymerase chain reaction is combined with reverse transcription polymerase chain reaction to quantify low abundance messenger RNA (mRNA), or miRNA, enabling a researcher to quantify relative gene expression at a particular time, or in a particular cell or tissue type.

In a real time PCR assay a positive reaction is detected by accumulation of a fluorescent signal. The Ct (cycle threshold) is defined as the number of cycles required for the fluorescent signal to cross the threshold (i.e. exceeds background level). Ct levels are inversely proportional to the amount of target nucleic acid in the sample (i.e. the lower the Ct level the greater the amount of target nucleic acid in the sample). Most real time assays undergo 40 cycles of amplification.

Ct-values <29 are strong positive reactions indicative of abundant target nucleic acid in the sample. Ct-values of 30-37 are positive reactions indicative of moderate amounts of target nucleic acid. Ct-values of 38-40 are weak reactions indicative of minimal amounts of target nucleic acid which could represent an infection state or environmental contamination.

The QPCR may be performed using chemicals and/or machines from a commercially available platform.

The QPCR may be performed using QPCR machines from any commercially available platform; such as Prism, geneAmp or StepOne Real Time FOR systems (Applied Biosystems), LightCycler (Roche), RapidCycler (Idaho Technology), MasterCycler (Eppendorf), iCycler iQ system, Chromo 4 system, CFX, MiniOpticon and Opticon systems (Bio-Rad), SmartCycler system (Cepheid), RotorGene system (Corbett Lifescience), MX3000 and MX3005 systems (Stratagene), DNA Engine Opticon system (Qiagen), Quantica qPCR systems (Techne), InSyte and Syncrom cycler system (BioGene), DT-322 (DNA Technology), Exicycler Notebook Thermal cycler, TL998 System (lanlong), Line-Gene-K systems (Bioer Technology), or any other commercially available platform.

The QPCR may be performed using chemicals from any commercially available platform; such as NCode EXPRESS VCR or EXPRESS VCR (Invitrogen), Taqman or SYBR green VCR systems (Applied Biosystems), Real-Time PCR reagents (Eurogentec), iTaq mix (Bio-Rad), qPCR mixes and kits (Biosense), and any other chemicals, commercially available or not, known to the skilled person.

The QPCR reagents and detection system may be probe-based, or may be based on chelating a fluorescent chemical into double-stranded oligonucleotides.

The QPCR reaction may be performed in a tube; such as a single tube, a tube strip or a plate, or it may be performed in a microfluidic card in which the relevant probes and/or primers are already integrated.

A Microfluidic card allows high throughput, parallel analysis of mRNA or miRNA expression patterns, and allows for a quick and cost-effective investigation of biological pathways. The microfluidic card may be a piece of plastic that is riddled with micro channels and chambers filled with the probes needed to translate a sample into a diagnosis. A sample in fluid form is injected into one end of the card, and capillary action causes the fluid sample to be distributed into the microchannels. The microfluidic card is then placed in an appropriate device for processing the card and reading the signal.

### Other Analysis Methods

The isolated RNA may be analysed by northern blotting. In one embodiment, the expression level of one or more miRNAs is determined by the northern blot technique.

A northern blot is a method used to check for the presence of a RNA sequence in a sample. Northern blotting combines denaturing agarose gel or polyacrylamide gel electrophoresis for size separation of RNA with methods to transfer the size-separated RNA to a filter membrane for probe hybridization. The hybridization probe may be made from DNA or RNA.

In yet another embodiment, the isolated RNA is analysed by nuclease protection assay.

The isolated RNA may be analysed by Nuclease protection assay.

Nuclease protection assay is a technique used to identify individual RNA molecules in a heterogeneous RNA sample extracted from cells. The technique can identify one or more RNA molecules of known sequence even at low total concentration. The extracted RNA is first mixed with antisense RNA or DNA probes that are complementary to the sequence or sequences of interest and the complementary strands are hybridized to form double-stranded RNA (or a DNA-RNA hybrid). The mixture is then exposed to ribonucleases that specifically cleave only single-stranded RNA but have no activity against double-stranded RNA. When the reaction runs to completion, susceptible RNA regions are degraded to very short oligomers or to individual nucleotides; the surviving RNA fragments are those that were complementary to the added antisense strand and thus contained the sequence of interest.

### Device

It is also an aspect of the present invention to provide a device for measuring the expression level of at least one miRNA in a sample, wherein said device comprises or consists of at least one probe or primer set for at least one miRNA selected from the group consisting of

a) miR-664; and
b) one or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

In another embodiment of the present invention a device for measuring the expression level of at least one miRNA in a sample is provided, wherein said device comprises or consists of at least one probe or primer set for at least one miRNA selected from the group consisting of
a) miR-382; and
b) one or more miRs selected from the group consisting of miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
c) one or more miRs selected from the group consisting of miR-592, miR-196b, miR-29b, miR-455-5p, miR-22, miR-204, miR-370, miR-338-3p, miR-99a*, miR-133b, miR-15a, miR-497, miR-29c*, miR-552, miR-181a, miR-660, miR-324-3p, miR-141, miR-874, miR-185, miR-99a, miR-545, miR-21*, miR-452, miR-143, miR-214*, miR-576-3p, miR-501-5p and miR-29c.
   wherein said device is used for characterising a sample.

It is preferred that at least one miR selected under b) is different from at least one miR selected under c).

It is also an aspect of the present invention to provide a device for measuring the expression level of at least one miRNA in a sample, wherein said device comprises or consists of at least one probe or primer set for
a) one or more miRs selected from the group consisting of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p, and miR-625; and
b) one or more miRs selected from the group consisting of miR-196b, miR-592, miR-545, miR-15a, miR-455-5p, miR-338-3p, miR-19b, miR-148a, miR-449a, miR-106b, miR-141, miR-18b, miR-379, miR-552, miR-29c, miR-181a, miR-193a-3p, and miR-636.
   wherein said device is used for characterising a sample.

It is also an aspect of the present invention to provide a device for measuring the expression level of at least one miRNA in a sample, wherein said device comprises or consists of at least one probe or primer set for
a) one or more miRs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

It is also an aspect of the present invention to provide a device for measuring the expression level of at least one miRNA in a sample, wherein said device comprises or consists of at least one probe or primer set for
a) one or more miRs selected from the group consisting of miR-17*, miR-22, miR-145, miR-155, miR-185, miR-196b, miR-204, miR-214*, miR-382, miR-449, miR-455, miR-501, miR-552, miR-545, miR-592 and miR-664.

A primer set of a given miR is a primer set capable of amplifying said miR in a PCR. Primers sets for miR are available from Applied Biosystems, United States.

In one embodiment, said device comprises or consists of at least one probe or primer set for miR-382 combined with at least one miRNA selected from the group consisting of the miRNAs mentioned in Tables 1, 2, 3 and 4, preferably selected from the group consisting of the miRNAs mentioned in Tables 5 and 6.

In another embodiment, said device comprises or consists of probes or primer sets for any of the miRs described herein above in the section “miRNA biomarkers of the present invention” or any of the classifiers described herein above in the section “miRNA classifier of the present invention”.

In one embodiment, said device comprises or consists of probes or primer set for one or more of miR selected from the group of miRNAs mentioned in Tables 1, 2, 3 4, 5 and 6.

In another embodiment, said device comprises or consists of probes or primer set for one or more of miRNAs selected from the group of miRNAs mentioned in Table 1 and one or more of miRNAs selected from the group of miRs mentioned in Table 2, wherein the miRNA selected from miRNAs mentioned in Table 1 is different from the miRNA selected from miRNAs of Table 2.

In another embodiment, said device comprises or consists of probes or primer set for one or more of miRNAs selected from the group of miRNAs mentioned in Table 3 and one or more of miRNAs selected from the group of miRs mentioned in Table 4, wherein the miRNA selected from miRNAs mentioned in Table 3 is different from the miRNA selected from miRNAs of Table 4.

In another embodiment, said device comprises or consists of probes or primer set for one or more of miRNAs selected from the group of miRNAs mentioned in Table 5 and one or more of miRNAs selected from the group of miRs mentioned in Table 6, wherein the miRNA selected from miRNAs mentioned in Table 5 is different from the miRNA selected from miRNAs of Table 6.

In another embodiment, said device comprises or consists of probes or primer set for one or more of miR-1, miR-17*, miR-22, miR-29a*, miR-29b, miR-145*, miR-185, miR-193b*, miR-204, miR-214*, miR-365, miR-382, miR-501-5p, miR-664, miR-1251, miR-15a, miR-148a, miR-155, miR-181a, miR-196b, miR-204, miR-214*, miR-338-3p, miR-449a, miR-449 b, miR-455-5p, miR-497, miR-545, miR-552, and miR-592.

In yet another embodiment said device comprises or consists of probes or primer set for one or more of miR selected from the group consisting of miR-1, miR-17*, miR-22, miR-29a*, miR-29b, miR-145*, miR-185, miR-193b*, miR-204, miR-214*, miR-365, miR-382, miR-497, miR-501-5p, miR-664, and miR-1251 and one or more of miR selected from the group consisting of miR-15a, miR-148a, miR-155, miR-181a, miR-196b, miR-204, miR-214*, miR-338-3p, miR-382, miR-449a, miR-449 b, miR-455-5p, miR-497, miR-545, miR-552, and miR-592, wherein said device comprises or consists of probes or primer set for at least two different miRs.

In another embodiment, said device comprises or consists of probes or primer set for one or more of the miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

In another embodiment, said device comprises or consists of probes or primer set for one or more of the miRNAs selected from the group consisting of miR-17*, miR-22, miR-145, miR-155, miR-185, miR-196b, miR-204, miR-214*, miR-382, miR-449, miR-455, miR-501, miR-552, miR-545, miR-592 and miR-664.

In another embodiment, said device comprises or consists of probes or primer set for one or more of miR-29b, miR-204, miR-214*, miR-382, and miR-497.

In one embodiment, the device may be used for distinguishing between cancer patients, for whom anti-angiogenic treatment has good efficacy from cancer patients for whom anti-angiogenic treatment has little or no efficacy.

In one embodiment said device comprises between 1 to 2 probes per miRNA to be measured, such as 2 to 3 probes, for example 3 to 4 probes, such as 4 to 5 probes, for example 5 to 6 probes, such as 6 to 7 probes, for example 7 to 8 probes, such as 8 to 9 probes, for example 9 to 10 probes, such as 10 to 15 probes, for example 15 to 20 probes, such as 20 to 25 probes, for example 25 to 30 probes, such as 30 to 40 probes, for example 40 to 50 probes, such as 50 to 60 probes, for example 60 to 70 probes, such as 70 to 80 probes, for example 80 to 90 probes, such as 90 to 100 probes or probe sets per miRNA of the present invention to be measured.

In another embodiment, said device has of a total of 1 probe or primer set for at least one miRNA to be measured, such as 2 probes, for example 3 probes, such as 4 probes, for example 5 probes, such as 6 probes, for example 7 probes, such as 8 probes, for example 9 probes, such as 10 probes, for example 11 probes, such as 12 probes, for example 13 probes, such as 14 probes, for example 15 probes, such as 16 probes, for example 17 probes, such as 18 probes, for example 19 probes, such as 20 probes, for example 21 probes, such as 22 probes, for example 23 probes, such as 24 probes, for example 25 probes, such as 26 probes, for example 27 probes, such as 28 probes, for example 29 probes, such as 30 probes, for example 31 probes, such as 32 probes, for example 33 probes, such as 34 probes, for example 35 probes, such as 36 probes, for example 37 probes, such as 38 probes, for example 39 probes, such as 40 probes, for example 41 probes, such as 42 probes, for example 43 probes, such as 44 probes, for example 45 probes, such as 46 probes, for example 47 probes, such as 48 probes, for example 49 probes, such as 50 probes or primer sets for at least one miRNA of the present invention to be measured.

It follows, that there may be one probe specific to a miRNA to be measured, or more than one probe specific to a miRNA to be measured—which may be called a probe set. In one embodiment, the device comprises 1 probe per miRNA to be measured, in another embodiment, said device comprises 2 probes, such as 3 probes, for example 4 probes, such as 5 probes, for example 6 probes, such as 7 probes, for example 8 probes, such as 9 probes, for example 10 probes, such as 11 probes, for example 12 probes, such as 13 probes, for example 14 probes, such as 15 probes per miRNA to be measured or analysed.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of

a) miR-664; and
b) one or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

In another embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of:
a) miR-382; and
b) one or more miRs selected from the group consisting of miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
c) one or more miR selected from the group consisting of miR-592, miR-196b, miR-455-5p, miR-370, miR-338-3p, miR-99a*, miR-133b, miR-15a, miR-497, miR-552, miR-181a, miR-141, miR-185, miR-545, miR-21*, miR-452, miR-143, miR-214* and miR-501-5p.

In one embodiment, the device may be a microarray chip; a QPCR Micro Fluidic Card; or may comprise QPCR tubes, QPCR tubes in a strip or a QPCR plate, comprising one or more probes for
a) one or more miRNAs selected from the group consisting of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146b-3p, miR-874, miR-1227, miR-29c*, miR-34b, miR-19b-1*, miR-100, miR-576-3p, miR-365, miR-660, miR-145, miR-505, miR-501-5p, and miR-625; and
b) one or more miRNAs selected from the group consisting of miR-196b, miR-592, miR-545, miR-15a, miR-455-5p, miR-338-3p, miR-19b, miR-148a, miR-449a, miR-106b, miR-141, miR-18b, miR-379, miR-552, miR-29c, miR-181a, miR-193a-3p, and miR-636.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of
a) miR-1, miR-17*, miR-22, miR-29a*, miR-29b, miR-145*, miR-185, miR-193b*, miR-204, miR-214*, miR-365, miR-382, miR-497, miR-501-5p, miR-664 and miR-1251; and
b) one or more miRNAs selected from the group consisting of miR-15a, miR-148a, miR-155, miR-181a, miR-196b, miR-338-3p, miR-449a, miR-449 b, miR-455-5p, miR-545, miR-552, and miR-592.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of
a) miR-1, MiR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

In one embodiment, the device may be a microarray chip; a QPCR Micro Fluidic Card; or may comprise QPCR tubes, QPCR tubes in a strip or a QPCR plate, comprising one or more probes for at least one miRNA and identified herein; selected from the group of
a) miR-17*, miR-22, miR-145, miR-155, miR-185, miR-196b, miR-204, miR-214*, miR-382, miR-449, miR-455, miR-501, miR-552, miR-545, miR-592 and miR-664.

### Computer Program Product

It is a further aspect of the invention to provide a computer program product having a computer readable medium, said computer program product comprising means for carrying out any of the herein listed miRNA classifiers, models and methods.

It is a further aspect of the invention to provide a system comprising means for carrying out any of the herein listed methods.

It is an aspect of the present invention to provide a system for predicting the efficacy of an anti-angiogenic treatment, said system comprising means for analysing the expression level of at least one miRNA in a sample obtained from an individual, wherein the expression level of said miRNAs is associated with colorectal cancer or other types of adenocarcinoma, wherein said at least one miRNA is selected from the group consisting of

a) miR-664; and
b) one or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, and miR-592.

In another embodiment of the present invention a system for predicting the efficacy of an anti-angiogenic treatment is provided, said system comprising means for analysing the expression level of at least one miRNA in a sample obtained from an individual, wherein the expression level of said miRNAs is associated with colorectal cancer or other types of adenocarcinoma, wherein said at least one miRNA is selected from the group consisting of
i) miR-382; and
ii) one or more miRNAs selected from the group consisting of miR-370, miR-193b*, miR-22, miR-497, miR-29c*, miR-145*, miR-501-5p, miR-146 b-3p, miR-29b, miR-185, miR-17*, miR-34b, miR-423-5p, miR-576-3p, miR-214*, miR-874, miR-190b, miR-152, miR-324-3p, miR-99a, miR-204, miR-455-5p, miR-143, miR-505, miR-660, miR-34a, miR-29a*, miR-100 and miR-151-3p; and
iii) one or more miRNAs selected from the group consisting of miR-592, miR-196b, miR-455-5p, miR-370, miR-338-3p, miR-99a*, miR-133b, miR-15a, miR-497, miR-552, miR-181a, miR-141, miR-185, miR-545, miR-21*, miR-452, miR-143, miR-214* and miR-501-5p.

It is an aspect of the present invention to provide a system for predicting the efficacy of an anti-angiogenic treatment, said system comprising means for analysing the expression level of at least one miRNA in a sample obtained from an individual, wherein the expression level of said miRNAs is associated with efficacy of an antiangiogenic treatment of a patient suffering from cancer, such as from colorectal cancer or other types of adenocarcinoma, wherein said at least one miRNA is selected from the group consisting of
a) miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p, and miR-625; and
b) one or more miRNAs selected from the group consisting of miR-196b, miR-592, miR-545, miR-15a, miR-455-5p, miR-338-3p, miR-19b, miR-148a, miR-449a, miR-106b, miR-141, miR-18b, miR-379, miR-552, miR-29c, miR-181a, miR-193a-3p, and miR-636.

It is an aspect of the present invention to provide a system for predicting the efficacy of an anti-angiogenic treatment, said system comprising means for analysing the expression level of at least one miRNA in a sample obtained from an individual, wherein the expression level of said miRNAs is associated with efficacy of an anti-agiogenic treatment of a patient suffering from cancer, for example from colorectal cancer or other types of adenocarcinoma, wherein said at least one miRNA is selected from the group consisting of
a) miR-1, miR-17*, miR-22, miR-29a*, miR-29b, miR-145*, miR-185, miR-193b*, miR-204, miR-214*, miR-365, miR-382, miR-497, miR-501-5p, miR-664 and miR-1251; and
b) one or more miRNAs selected from the group consisting of miR-15a, miR-148a, miR-155, miR-181a, miR-196b, miR-338-3p, miR-449a, miR-449 b, miR-455-5p, miR-545, miR-552, and miR-592.

It is an aspect of the present invention to provide a system for predicting the efficacy of an anti-angiogenic treatment, said system comprising means for analysing the expression level of at least one miRNA in a sample obtained from an individual, wherein the expression level of said miRNAs is associated with efficacy of an anti-angiogenic treatment of a patient suffering from cancer, for example from colorectal cancer or other types of adenocarcinoma, wherein said at least one miRNA is selected from the group consisting of
a) miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552, miR-592, and miR-664.

It is an aspect of the present invention to provide a system for predicting the efficacy of an anti-angiogenic treatment, said system comprising means for analysing the expression level of at least one miRNA in a sample obtained from an individual, wherein the expression level of said miRNAs is associated with efficacy of an anti-agiogenic treatment of a patient suffering from cancer, for example from colorectal cancer or other types of adenocarcinoma, wherein said at least one miRNA is selected from the group consisting of
a) miR-17*, miR-22, miR-145, miR-155, miR-185, miR-196b, miR-204, miR-214*, miR-382, miR-449, miR-455, miR-501, miR-552, miR-545, miR-592 and miR-664.

In another aspect, the present invention provides a computer program product having a computer readable medium, said computer program product providing a system for predicting the efficacy of an anti-angiogenic treatment of an individual, said computer program product comprising means for carrying out any of the steps of any of the methods as disclosed herein.

In another aspect, the present invention provides a system as disclosed herein wherein the data is stored, such as stored in at least one database.

### Kit-of-Parts

It is also an aspect to provide a kit-of-parts comprising the device according to the present invention, and at least one additional component.

In one embodiment, the additional component may be used simultaneously, sequentially or separately with the device.

In one embodiment, said additional component comprises means for extracting RNA such as miRNA from a sample; reagents for performing microarray analysis and/or reagents for performing QPCR analysis.

In another embodiment, said kit may comprise instructions for use of the device and/or the additional components.

In a further embodiment, said kit comprises a computer program product having a computer readable medium as detailed herein elsewhere.

### Anti-Angiogenic Treatment

The methods according to the present invention relates to predicting the efficacy of an anti-angiogenic treatment. The anti-angiogenic treatment may be any treatment, which as the primary objective has inhibition of the formation of new blood vessels from pre-existing vessels.

A preferred anti-angiogenic treatment according to the present invention is treatment with an inhibitor of Vascular endothelial growth factor (VEGF) or VEGF receptor.

Said VEGF may be any VEGF, preferably a VEGF selected from the group consisting of VEGF-A, PIGF, VEGF-B, VEGF-C and VEGF-D. More preferably, said VEGF is selected from the group consisting of human VEGF-A, human PIGF, human VEGF-B, human VEGF-C and human VEGF-D.

The VEGF receptor may any VEGF receptor, preferably a VEGF receptor selected from the group consisting of VEGFR-1 (also denoted Flt-1), VEGFR-2 (also denoted KDR/Flk-1) and VEGFR-3, more preferably selected from the group consisting of human VEGFR-1, human VEGFR-2 and human VEGFR-3.

The inhibitor may for example be a small organic molecule inhibitor. The inhibitor may also be a biologic macromolecule inhibitor, e.g. an antibody or an antibody fragment. Non-limiting examples of anti-angiogenic treatment includes for example treatment with one of more of the following compounds:
i) lenvatinib of the structure

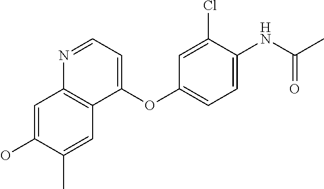 [SMILES: CC(=O)Nc1ccc(Oc2ccnc3cc(O)c(C)cc23)cc1Cl]

and pharmaceutically acceptable salts thereof;
ii) Motesanib of the structure

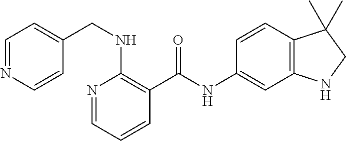 [SMILES: CC1(C)CNc2cc(NC(=O)c3cccnc3NCc3ccncc3)ccc21]

or pharmaceutically acceptable salts thereof
iii) Pazopanib of the structure

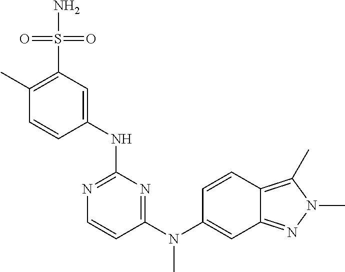

or pharmaceutically acceptable salts thereof,
iv) lapatinib of the structure

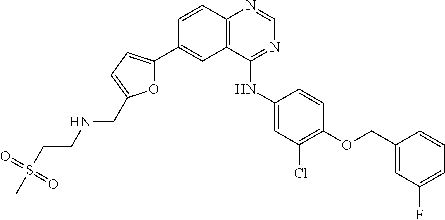 [SMILES: CS(=O)(=O)CCNCc1ccc(-c2ccc3ncnc(Nc4ccc(OCc5cccc(F)c5)c(Cl)c4)c3c2)o1]

or pharmaceutically acceptable salts thereof (Tykerb),
v) sunitinib (Sutent) of the structure

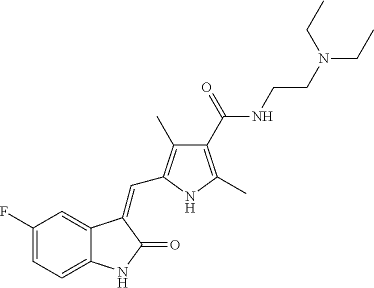 [SMILES: CCN(CC)CCNC(=O)c1c(C)[nH]c(/C=C2\C(=O)Nc3ccc(F)cc32)c1C]

or pharmaceutically acceptable salts thereof,
vi) sorafenib (Nexavar) of the structure

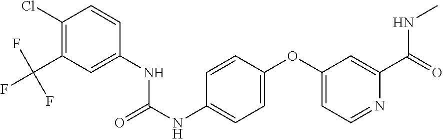 [SMILES: CNC(=O)c1cc(Oc2ccc(NC(=O)Nc3ccc(Cl)c(C(F)(F)F)c3)cc2)ccn1]

or pharmaceutically acceptable salts thereof,
vii) axitinib of the structure

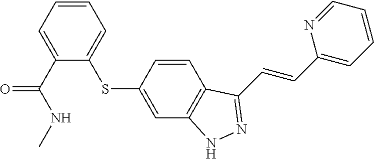 [SMILES: CNC(=O)c1ccccc1Sc1ccc2c(/C=C/c3ccccn3)n[nH]c2c1]

or a pharmaceutically acceptable salts thereof
viii) Aflibercept (Zaltrap) (CAS number 862111-32-8)
ix) Regorafenib (Stivarga) of the structure

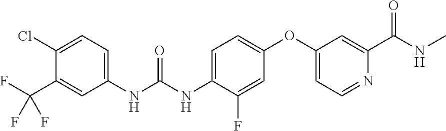 [SMILES: CNC(=O)c1cc(Oc2ccc(NC(=O)Nc3ccc(Cl)c(C(F)(F)F)c3)c(F)c2)ccn1]

x) Antibodies or antigen binding fragments thereof specifically binding either VEGF or VEGF receptors for example
   a. Ranibizumab (Lucentis) (Genentech, United States) or
   b. Bevacizumab (Avastin) (Genentech, United States)

### Chemotherapeutic Treatment

Chemotherapeutic treatment according to the present invention is treatment of a cancer patient with chemotherapy. Chemotherapeutic treatment may thus be treatment of a cancer patient with one or more cytotoxic agents. For example said cytotoxic agents may be capecitabine and oxaliplatin.

### Examples

### Example 1

### Patients

Patients with metastatic colorectal cancer (mCRC) treated with first line bevacizumab (Bev) and chemotherapy (capecitabine and oxaliplatin (CapOx)) from seven Departments of Oncology in Denmark were retrospectively included. Inclusion criteria were biopsy-confirmed adenocarcinoma of the colon or rectum with distant metastases, and first line systemic treatment for metastatic disease with CapOx and bevacizumab (CapOxBev). Exclusion criteria were: other malignancy during the past 5 years or discovered during treatment or follow-up, uncertainty about primary tumor location, primary tumor in appendix, endocrine histology, and CapOxBev given explicitly as neo-adjuvant or adjuvant treatment.

Data about baseline characteristics, treatment, and disease progression was extracted from patient records and electronic databases at each hospital. Pathology data and survival status was extracted from national databases using the unique Central Person Registration number assigned to every Danish citizen. This allowed for complete information about pathologic diagnoses and updated survival status for every patient. The endpoints time to disease progression (TTP) and overall survival (OS) was measured from date of start of CapOxBev treatment to date of first disease progression and date of death, respectively.

### Collection of CRC Tissue

Only tissue samples from primary tumors were included. If possible, tissue from tumor resections was used. If the primary tumor had not been resected or the primary tumor had been treated with chemo- or radiotherapy previously, tissue from the diagnostic biopsy was used. CRC tissue was collected at time of operation and diagnostic biopsy at the individual Departments of Pathology. The cancer samples were routinely stored as formalin-fixed paraffin-embedded (FFPE) samples.

An experienced gastro-intestinal pathologist selected relevant tissue blocks for the study. Before sectioning for miRNA analysis, one 3 μm section was cut and stained with Hematoxylin and Eosin (HE) according to standard procedures. This HE-stained section was reviewed by the same pathologist and scored for tumor cell content, inflammation, tumor budding, necrosis, and fibrosis. Only samples approved by the pathologist were sectioned for miRNA analysis. From each approved FFPE sample 3 sections of 10 μm were cut for miRNA analysis.

RNA purification and determination of miRNA expression RNA was purified with the miRNeasy FFPE Kit from Qiagen using the manufacturer's instructions. The non-human miRNA ath-miR-159a was added to each sample before purification as a positive control to monitor RNA isolation and as a positive control for real-time amplification.

The TaqMan® Human MicroRNA assay using A Cards v2.0 and B Cards v3.0 (Part Number 4400238, Applied Biosystems, United States) was used in order to select the most significant and interesting miRNAs in CRC tissue of patients with CRC treated with CapOxBev. This method used a set of two pre-configured micro fluidic cards that enables quantization of 754 human miRs. Included on each array were three TaqMan MicroRNA assay endogenous controls to aid in data normalization and one TaqMan® MicroRNA assay not related to human as a negative control. The instructions from Applied Biosystems were followed in all details including the use of pre-amplification (https://products.appliedbiosystems.com).

The Ct-value was determined, wherein Ct-value was the number of cycles required for the fluorescent signal to cross the background level. Thus a lower Ct-value indicates a higher level of miRNA. To ease understanding, the variable 40 minus the Ct-value was used for further analysis. Thus a Ct-value of 40 or higher is considered no expression.

### Statistical Analysis

Raw miRNA expression values were checked for outliers and data was corrected using spike-in values. In a univariate selection method, each miRNA was related to time-to-disease progression (TTP) and overall survival (OS) using Cox proportional hazards model (e.g. as described in [1, 2]) (threshold p<0.001). TTP was defined as time from start of first line treatment with CapOxBev to disease progression, either Response Evaluation Criteria in Solid Tumors (RECIST) progression or clinical progression. OS time was defined as time from start of first line treatment with CapOxBev to death of any cause. Candidate miRNAs were tested using Akaike's Information Criterion (e.g. described in [3]) in a multivariate analysis corrected for age, sex, histology, number of metastatic sites, primary location, and prior adjuvant treatment. The combined predictive value of the miRNAs from the multivariate model was illustrated in a Kaplan-Meier plot by calculating a prognostic index (PI) for each patient. This was done by multiplying each of the significant miRNA expressions (xi) with their corresponding estimated coefficient (βi) from the Cox regression and summing over all terms, i.e. PI=xi βi. We then divided the patients into two groups, those with PI below the median PI and those with PI above the median PI. The Kolmogorov-Smirnov test (as described in [4 to 7]) was utilized to investigate possible differences in individual miRNA expression caused by chemo-/radiotherapy pre-treatment, low tumor cell content (<20%), and biopsied sample. The statistical software package R (www.r-project.org) was used for all analyses. This is obtainable from The R Foundation for Statistical Computing, Austria.

### Patient Samples

383 patients were included from 7 hospitals based on the inclusion/exclusion criteria described above. FFPE tissue blocks from primary tumors with acceptable tumor content could be retrieved and sectioned for 212 patients. Patients covered a broad range of age, sex and performance status.

Quality Assessment and Establishment of Final miRNA Data Set

212 patient samples were purified and analyzed on the TaqMan® Human MicroRNA assay using A Cards v2.0 and B Cards v3.0 (Part Number 4400238, Applied Biosystems, United States). 10 samples were found to have poor RNA purification quality which also influenced the number of miRNAs detected, and these were re-purified from new sections and re-analyzed. All of these sample results were acceptable after re-analysis. The dataset was analyzed for outliers and 9 sample results were removed as outliers based upon a low number of detectable miRNAs.

There was a strong linear correlation between higher than planned spike-in cycle threshold (Ct) value (>22) and both high mean Ct-value and low number of detectable miRNAs, so for each Ct-value in samples with spike-in Ct-values above 22 we subtracted 0.28×(SpikeIn-22). There was a significant association between sample age and mean Ct-value and number of detectable miRNAs, but the effect size was negligible.

Biopsy-origin and low tumor cell content, but not pre-treatment, were found to influence individual miRNA expression.

There is no consensus about normalization of miRNA array data. We have therefore tried different normalization techniques: A) Raw CV (Raw values but the optimal cut-off point for the univariate p-values is found by cross validation (e.g. described in [8]); B) Raw ORG (This is an analysis with a fixed significance cut-off point decided prior to the analysis); C) Endo CV (Normalization with endogenous controls RNU44 and RNU48 and cut-off found by cross validation); D) Quan CV (Quantile normalization, e.g. described in [9], using cross validation for the cut-off); E) Quan Org (Quantile normalization using a fixed cut-off); F) 120 CV (Normalization with the mean of the 120 most expressed miRNAs and cut-off found by cross validation); and G) Rank (Rank normalization where the cut-off is found by cross validation). After outlier removal and spike-in correction, 203 sample results remained in the dataset. All of these had data available for OS but only 192 had data for TTP.

For the calculations relating miRNA expression to outcome, expression values were changed to the value “40 minus Ct-value” so that a higher value would be equal to higher miRNA expression. This was done to ease understanding of the results, since higher Ct-values correspond to lower expression. “40 minus Ct-value” is also denoted “40-Ct-value” herein.

Correlation with Time to Disease Progression and Overall Survival

In the univariate selection method a total of 116 miRNAs were selected as indicative of TTP (see Table 1) and a total of 74 miRNAs were selected as indicative of OS (see Table 2) (p-value <0.05). 18 miRNAs were highly predictive of TTP and 4 miRNAs were highly predictive of OS (threshold p≦0.001). The univariate selection method correlates expression level (40 minus Ct-value) to either TTP or OS. The full list of univariately significant miRNAs (Raw values) related to TTP are given in Table 1 and to OS in Table 2. The full list of univariately significant miRNAs (Quantile normalization) related to TTP are given in Table 3 and to OS in Table 4.

In the multivariate analysis the result of the univariate analysis was corrected for age, sex, histology, number of metastatic sites, primary location, and prior adjuvant treatment in order to select only miRNAs wherein the expression level is independently indicative of TTP or OS. Table 5 shows the list of significant miRNAs related to TTP (using the different normalization techniques and raw values) in the multivariate analysis corrected for age, sex, histology, number of metastatic sites, primary location, and prior adjuvant treatment.

Table 6 shows the list of significant miRNAs related to OS (using the different normalization techniques and raw values) in the multivariate analysis corrected for age, sex, histology, number of metastatic sites, primary location, and prior adjuvant treatment.

TABLE 5
MiRNAs significantly and independently associated with TTP after stepwise selection.
Results are shown as hazard ratio (HR) per inter-quartile range increase.

| Normalization | A | B | C | D | E | F | G |
|---|---|---|---|---|---|---|---|
| MIR | HR (95% CI) | HR (95% CI) | HR (95% CI) | HR (95% CI) | HR (95% CI) | HR (95% CI) | HR (95% CI) |
| miR-1 | | | | | | 0.81 (0.64-1.02) | |
| miR-17* | | | 0.80 (0.69-0.94) | 0.71 (0.57-0.87) | | 0.71 (0.55-0.91) | |
| miR-22 | 1.70 (1.19-2.43) | 1.20 (0.93-1.55) | | | | 1.29 (0.99-1.68) | |
| miR-29a* | | | | | | 1.42 (0.92-2.18) | |
| miR-29b | 0.56 (0.39-0.80) | 0.56 (0.40-0.80) | | | | 0.67 (0.42-1.06) | |
| miR-145* | 0.67 (0.46-0.97) | 0.71 (0.49-1.03) | | 0.77 (0.58-1.02) | 0.66 (0.50-0.87) | | |
| miR-185 | | | | | 0.88 (0.77-1.00) | | |
| miR-193b* | 0.71 (0.47-1.08) | 0.62 (0.43-0.89) | | 0.77 (0.58-1.02) | 0.78 (0.61-0.99) | 0.73 (0.55-0.97) | |
| miR-204 | | | 0.79 (0.64-0.98) | 0.71 (0.56-0.89) | | 0.81 (0.66-1.01) | |
| miR-214* | | | | | | 0.75 (0.57-0.99) | |
| miR-365 | | | 0.70 (0.57-0.87) | | | | |
| miR-382 | 0.67 (0.42-1.07) | | | | | | |
| miR-497 | | | | | | 0.73 (0.53-0.99) | 0.74 (0.61-0.90) |
| miR-501-5p | | | | | | 0.77 (0.60-0.99) | |
| miR-664 | | | | 1.33 (1.10-1.62) | | 1.22 (1.02-1.46) | |
| miR-1251 | | | | | | | 1.20 (1.01-1.42) |

Abbreviations: HR, hazard ratio; IQRI, inter-quartile range increase.

TABLE 6
MiRNAs significantly and independently associated with OS after stepwise selection.
Results are shown as hazard ratio (HR) per inter-quartile range increase.

| Normalization | A | B | C | D | E | F | G |
|---|---|---|---|---|---|---|---|
| MiR | HR (95% CI) | HR (95% CI) | HR (95% CI) | HR (95% CI) | HR (95% CI) | HR (95% CI) | HR (95% CI) |
| miR-15a | | | | 0.60 (0.41-0.88) | 0.54 (0.37-0.80) | | |
| miR-22 | | | | 1.25 (0.98-1.59) | | | |
| miR-29b | 0.61 (0.41-0.90) | | | | | 0.59 (0.44-0.79) | |
| miR-148a | | | | 1.23 (0.94-1.61) | 1.33 (0.96-1.85) | | |
| miR-155 | | | | | 1.36 (0.89-2.07) | | |
| miR-181a | 1.46 (0.92-2.32) | | | | | | |
| miR-196b | 0.67 (0.50-0.89) | 0.74 (0.59-0.93) | | 0.68 (0.50-0.93) | | 0.73 (0.52-1.03) | |
| miR-204 | | | 0.61 (0.47-0.78) | 0.54 (0.40-0.74) | 0.45 (0.32-0.64) | 0.60 (0.45-0.80) | |
| miR-214* | | | | | | 0.69 (0.54-0.89) | |
| miR-338-3p | | | | | 1.33 (0.95-1.86) | | |
| miR-382 | 0.48 (0.31-0.76) | 0.65 (0.50-0.84) | | 0.65 (0.48-0.87) | 0.79 (0.59-1.05) | | |
| miR-449a | | | | 1.40 (1.07-1.84) | | 1.88 (1.48-2.40) | |
| miR-449b | | | | | 1.46 (1.12-1.90) | | |
| miR-455-5p | 1.47 (0.96-2.24) | | | | | | |
| miR-497 | | | | | | | 0.65 (0.54-0.77) |
| miR-545 | | | | 0.57 (0.39-0.83) | 0.62 (0.42-0.91) | 0.77 (0.59-1.01) | |
| miR-552 | | | 0.76 (0.63-0.93) | | 0.71 (0.53-0.94) | 0.78 (0.64-0.94) | 0.84 (0.72-0.98) |
| miR-592 | | | | | | | 0.75 (0.56-0.99) |

Abbreviations: HR, hazard ratio; CI, confidence interval.

Sixteen miRNAs (miR-1, miR-17*, miR-22, miR-29a*, miR-29b, miR-145*, miR-185, miR-193b*, miR-204, miR-214*, miR-365, miR-382, miR-497, miR-501-5p, miR-664, and miR-1251) measured in archival FFPE samples of primary CRC tumors from patients treated with 1.-line CapOxBev were predictors of TTP (i.e. predictive biomarkers). Eighteen miRNAs (miR-15a, miR-22, miR-29b, miR-148a, miR-155, miR-181a, miR-196b, miR-204, miR-214*, miR-338-3p, miR-382, miR-449a, miR-449b, miR-455-5p, miR-497, miR-545, miR-552, and miR-592) were predictors of OS (i.e. prognostic biomarkers). Six miRNAs (miR-22, miR-29b, miR-204, miR-214*, miR-382, and miR-497) were associated with both TTP and OS. For most of the miRNAs lower expression predicted shorter TTP and shorter OS.

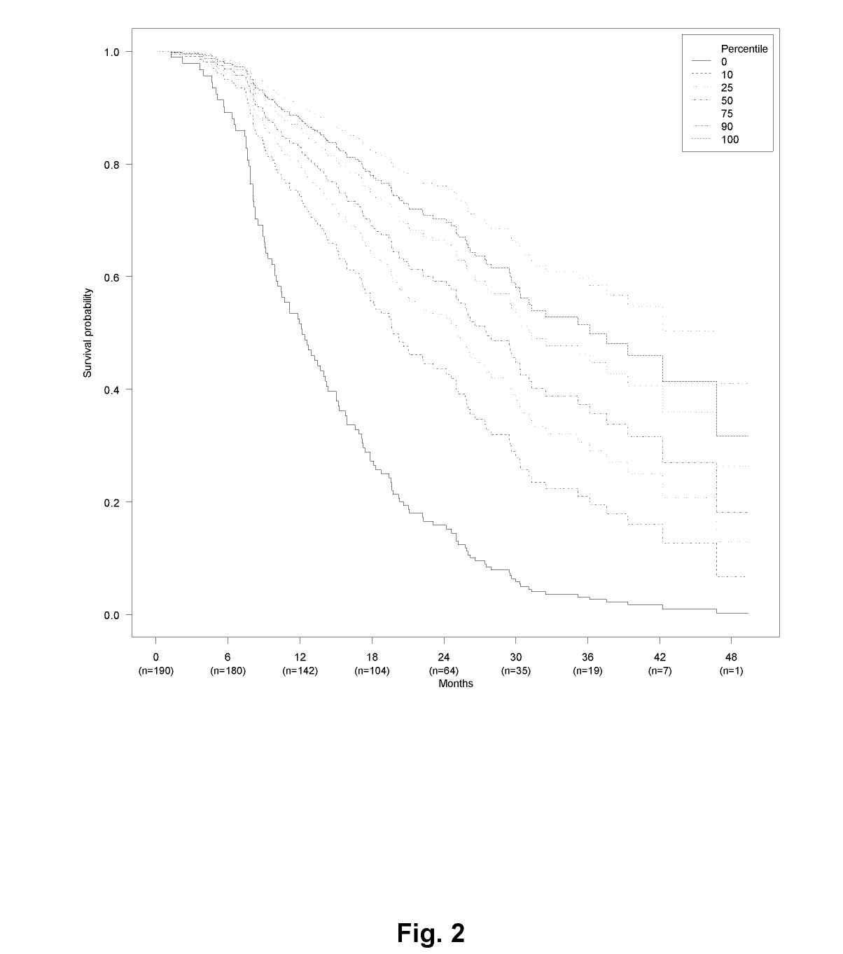

MiR-382 was the most significant predictor of both TTP and OS in univariate analysis, and it has predictive value in the multivariate model for OS. Increasing expression of miR-382 predicted improved survival and low expression of miR-382 predicted short OS, Table 6 and FIG. 2.

### Example 2

### Validation Study

A two-armed validation study is performed, each encompassing 200-250 new FFPE tumor samples from patients with metastatic CRC included from 5 hospitals in Denmark. In one arm patients with metastatic CRC who received CapOxBev are included, as described in Example 1, and in the other arm patients with metastatic CRC who received CapOx chemotherapy only are included. MiRNAs validated in the CapOxBev arm only are preferred as they are likely to be related to the efficacy of bevacizumab addition to chemotherapy.

CRC tissue samples are collected as described herein above in Example 1 and RNA is isolated as described herein above in Example 1.

30 different miRNAs with the lowest p-values determined as described in Example 1 are analysed using a miRNA array from Fluidigm BioMark System. This array system can perform 2,304 simultaneous real-time PCR experiments running gold-standard TaqMan® assays in nanolitre quantities. The 30 miRNAs will primarily be selected based upon their performance in the multivariate analyses (shown in Tables 5 and 6), secondarily, p-values from the univariate analyses will be used (Tables 1, 2, 3 and 4).

### Example 3

### Patients

400 patients with metastatic CRC treated with chemotherapy (capecitabine and oxaliplatin) and with bevacizumab (CapOxBev) are included from the two cohorts described in Example 1 and 2.

5-20 different miRNAs (selected as described in Example 2) are analysed using miRNA PCR method with reagents from TaqMan®. Thus primers useful for amplification of the selected miRNAs are as provided by Applied Biosystems, United States.

### Example 4

### Patients

400 patients with metastatic CRC treated with chemotherapy (capecitabine and oxaliplatin) and with bevacizumab (CapOxBev) are included from the two cohorts described in Example 1 and 2.

The localization and amount of the miRNA expression in tumor biopsies (FFPE CRC tissue) of up to 5 different miRNAs (selected as described in Example 2) are analysed using in situ hybridization methods with reagents from Exiqon®, Vedbaek, Denmark.

### Example 5

### Methods

### Study Design

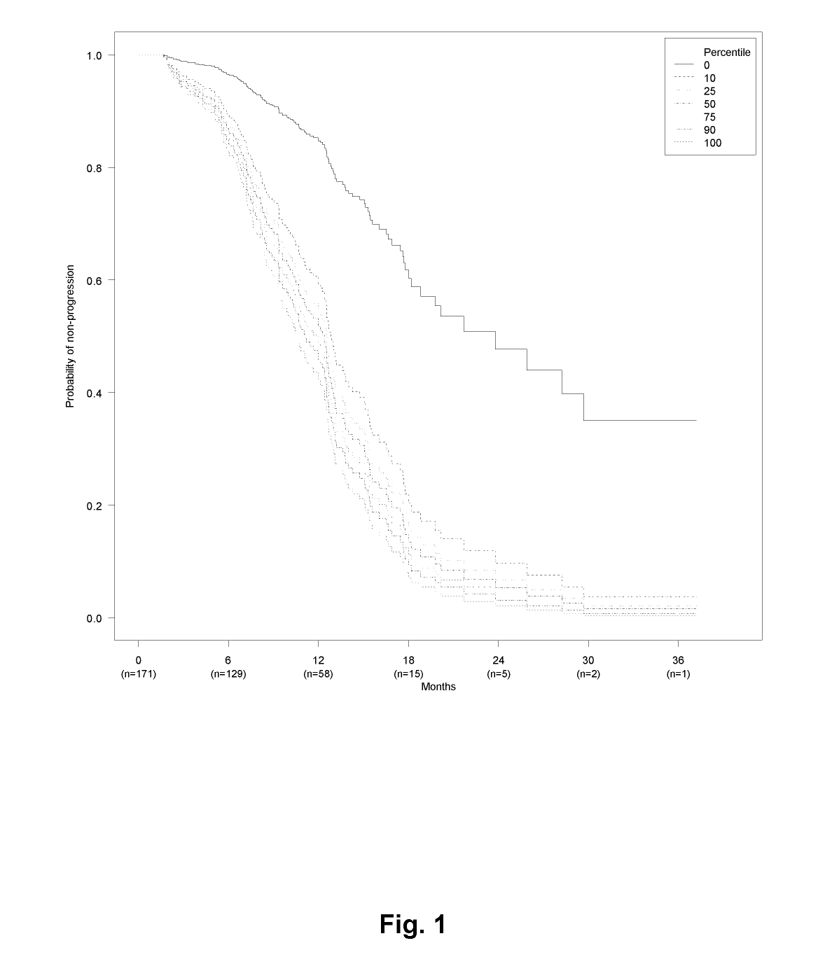

The study was conducted in three steps (FIG. 1). In the first step, we identified candidate miRNAs which could be predictive of bevacizumab efficacy. This step was described in Example 1. In step two, we measured the most promising miRNAs from step one in the discovery cohort again and validate these findings in a similar cohort of patients treated with CapOxBev. In step three, we measured the same candidate miRNAs in a cohort of patients treated with CapOx chemotherapy alone and compared with the combined cohort of CapOxBev treated patients, to identify miRNAs that are predictive of outcome for treatment with CapOx with bevacizumab but not CapOx chemotherapy alone. Samples from all three cohorts were measured together on the same platform in a randomized order for steps 2 and 3.

### Patients

We included three patient cohorts in the validation step. The discovery cohort was described in Example 1. Samples from the discovery study were used if outcome information was complete for both time to disease progression (TTP) and overall survival (OS) and if RNA quality was acceptable (n=155). A validation cohort of 119 patients treated with CapOxBev was included from 6 Danish departments of oncology. These patients were treated in the same period and were included using the same criteria as for the discovery cohort. A third cohort of 125 patients treated with CapOx alone were included. These patients were treated with the same chemotherapy (CapOx) but without bevacizumab in the period before bevacizumab was approved in Denmark. The third cohort was included partly from a randomized study conducted in the period and partly from Herlev University Hospital.

### Samples

Formalin-fixed paraffin-embedded (FFPE) tissue blocks from primary tumors were used and sectioning and purification was performed as described in Example 1 for the discovery cohort. The discovery samples that were reused were already purified. The purification of the new samples was done in a randomized order.

### MicroRNA Analyses

We selected the 22 miRNAs with the most significant correlation with TTP and OS from the “Discovery Study” described in Example 1, for further analyses (table 2.1). The main criterion for selection was significant correlation with outcome in multivariate analyses for more than one type of normalization. After purification and pre-amplification, the 22 miRNAs were measured in duplicate on custom Low Density Array cards (cLDA) from Applied Biosystems using Taqman microRNA PCR assays with samples from 8 patients on every card. A non-human control miRNA was spiked-in for quality control. The samples from the three cohorts were analyzed in a randomized order. MiRNA expression was measured as PCR cycle threshold (Ct) values.

### Statistical Analysis

For the calculations relating miRNA expression to outcome, expression values were changed to the value “40 minus Ct” so that a higher value would be equal to higher miRNA expression, as described in Example 1. In all cohorts, the correlation between miRNA expression and TTP and OS was tested using Cox proportional hazards models. Hazard ratio (HR) per interquartile range increase for miRNA expression was used as a measure of magnitude of the correlation between each miRNA and outcome. A HR above 1 equals worse outcome (shorter TTP and shorter OS) for increased miRNA expression and a HR below 1 equals improved outcome (longer TTP and longer OS) for increased miRNA expression. All miRNAs were tested univariately and the miRNAs which were most significantly correlated with outcome were included in a multivariate analysis with backwards elimination using Akaike's Information Criterion. All analyses were done on both raw and mean normalized expression values. Because we discovered during our analyses that the location of the primary tumor could be predictive of bevacizumab efficacy, with improved outcome with bevacizumab treatment in patients with primary tumors originating in the sigmoid colon and rectum, we also performed all the analyses stratified for primary tumor location group (sigmoid colon and rectum versus caecum to descending colon). A two-sided p-value <0.05 was considered statistically significant.

### Results

### MiRNA Predictors of Outcome in CapOxBev and CapOx Treated Cohort

14 miRNAs were significantly correlated with either TTP or OS for CapOxBev treated patients in univarate analysis of mean-normalized expression values (Table 2.2). Five of the 7 miRNAs that were significant in the discovery cohort were also significant with similar hazard ratios in the validation cohort. FIG. 2.2 shows the Kaplan-Meier curves for overall survival according to miR-664 expression above and below median. Only one miRNA was significantly correlated with outcome in the CapOx treated cohort. MiR-193b.5p predicted longer TTP with a univariate hazard ratio per interquartile range increase of 0.771 and a p-value of 0.03 in univariate- and 0.02 in multivariate analysis.

### MiRNAs Showing Interaction Between Correlation to Outcome and Bevacizumab Treatment

Three miRNAs showed a significant interaction between correlation to outcome and treatment group: miR-382.5 for TTP, and miR-455.5p and miR-664.3p for OS (Table 2.3). Five other miRNAs showed a trend (p<0.1) towards an interaction. Most of these 8 miRNAs were significantly correlated with outcome in the CapOxBev cohort (Table 2.2).

### Analyses Stratified by Primary Tumor Location

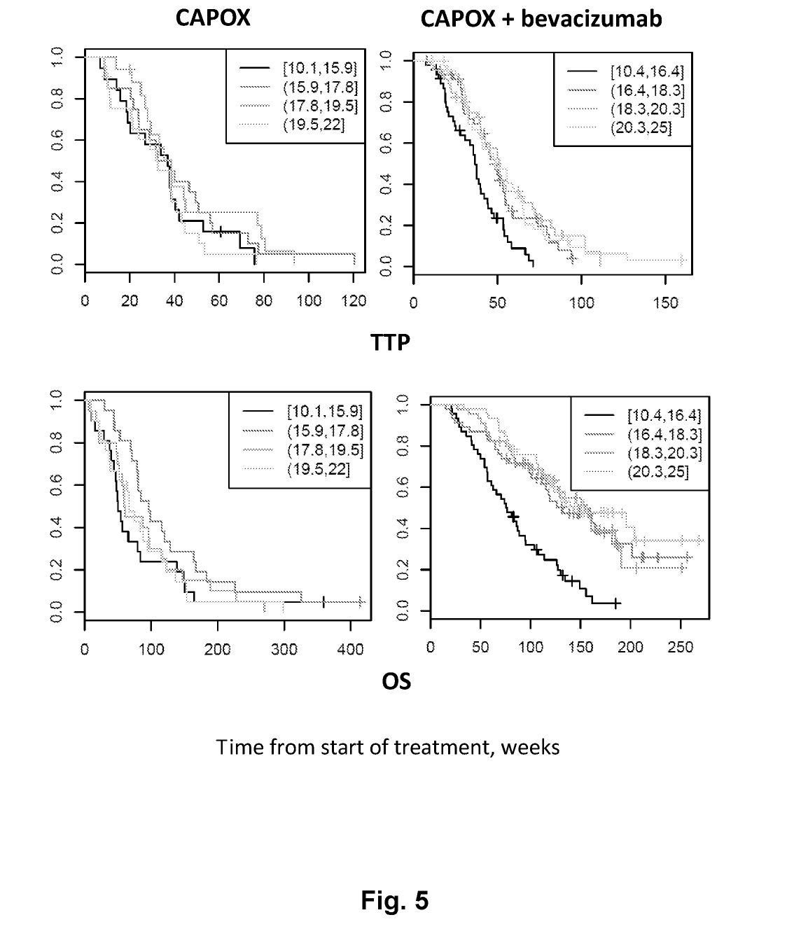

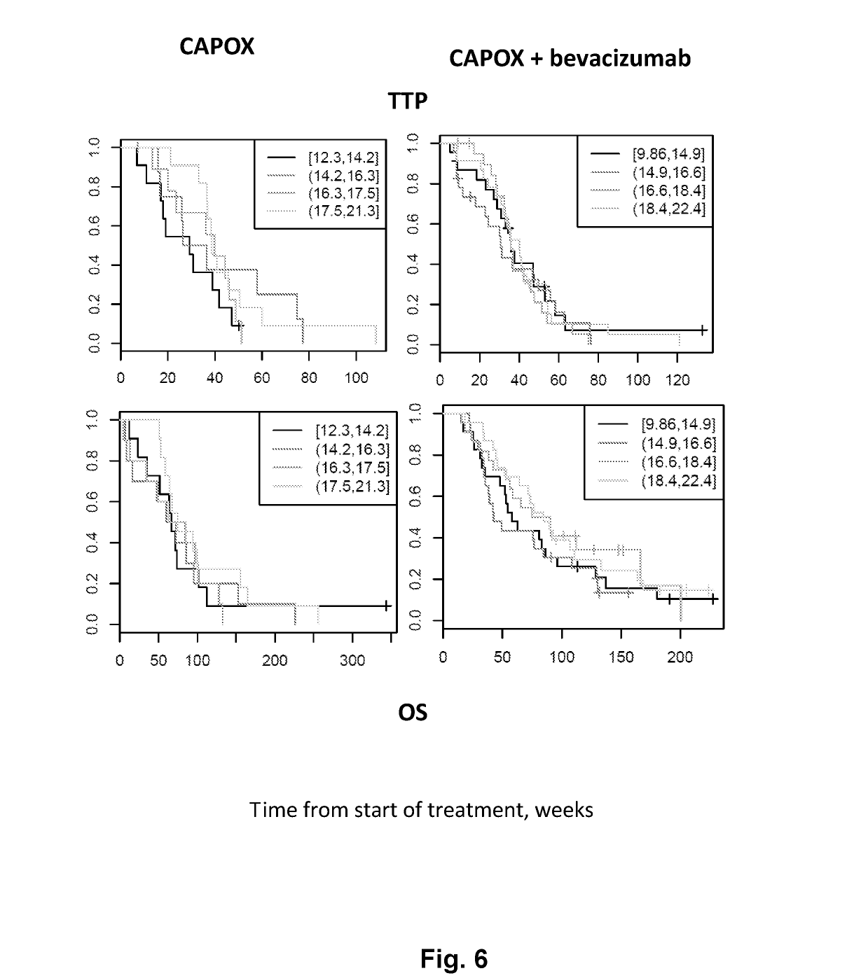

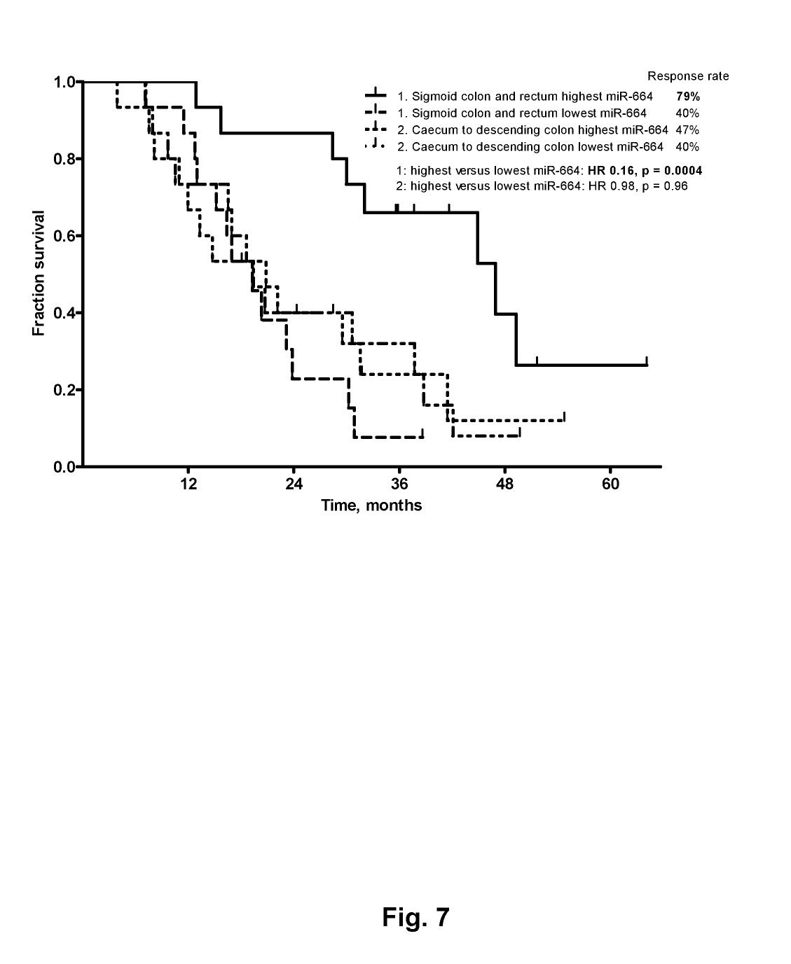

In Tables 2.4 and 2.5, results are stratified by primary tumor location. For some miRNAs, such as miR-155, miR-185, and miR-592, the correlation to outcome and significance levels were similar between the two location groups. Yet, some miRNAs were significant in only one or the other location group. MiR-664 showed differential correlation to outcome according to primary tumor location. The 75% highest expression values of miR-664 clearly predicted a longer TTP and OS in patients with primary tumors originating in the sigmoid- and rectosigmoid colon and rectum (FIG. 5). This difference was not seen for patients with primary tumors originating in the part of the colon stretching from the caecum to the descending colon (FIG. 6). When comparing the 15 patients with the highest and lowest expression values of miR-664 in the two location groups, only patients with primary tumors originating in the sigmoid- and rectosigmoid colon and rectum showed improved outcome with high versus low miR-664 expression with a response rate of 79% versus 40% and a HR for OS=0.16, p=0.0004 (FIG. 7).

### Discussion

We have identified a panel of interesting miRNAs in FFPE tissue from colorectal cancer associated to outcome in patients with mCRC treated with CapOxBev but not with CapOx alone, and interesting also a few predictive miRNAs with differential effects according to location of the primary tumor. These miRNAs have the potential as new predictive biomarkers for patients with mCRC (also described in [10]).

TABLE 8
MicroRNAs (miRNAs) selected
for further validation

| MiRNA | MiRNA | Mature |
|---|---|---|
| assay | miRBase | miRNA |
| name¹ | name² | sequence¹ |
| hsa-miR-1 | hsa- | UGGAAUGUAAAGAAGUAUGUAU |
| | miR-1 | |
| hsa-miR- | hsa-miR- | UAGCAGCACAUAAUGGUUUGUG |
| 15a | 15a-5p | |
| hsa-miR- | hsa-miR- | ACUGCAGUGAAGGCACUUGUAG |
| 17* | 17-3p | |
| hsa-miR- | hsa-miR- | AAGCUGCCAGUUGAAGAACUGU |
| 22 | 22-3p | |
| hsa-miR- | hsa-miR- | UAGCACCAUUUGAAAUCAGUGUU |
| 29b | 29b-3p | |
| hsa-miR- | hsa-miR- | GGAUUCCUGGAAAUACUGUUCU |
| 145* | 145-3p | |
| hsa-miR- | hsa-miR- | UUAAUGCUAAUCGUGAUAGGGGU |
| 155 | 155-5p | |
| hsa-miR- | hsa-miR- | UGGAGAGAAAGGCAGUUCCUGA |
| 185 | 185-5p | |
| hsa-miR- | hsa-miR- | CGGGGUUUUGAGGGCGAGAUGA |
| 193b* | 193b-5p | |
| hsa-miR- | hsa-miR- | UAGGUAGUUUCCUGUUGUUGGG |
| 196b | 196b-5p | |
| hsa-miR- | hsa-miR- | UUCCCUUUGUCAUCCUAUGCCU |
| 204 | 204-5p | |
| hsa-miR- | hsa-miR- | UGCCUGUCUACACUUGCUGUGC |
| 214* | 214-5p | |
| hsa-miR- | hsa-miR- | UCCAGCAUCAGUGAUUUUGUUG |
| 338-3p | 338-3p | |
| hsa-miR- | hsa-miR- | GAAGUUGUUCGUGGUGGAUUCG |
| 382 | 382-5p | |
| hsa-miR- | hsa-miR- | UGGCAGUGUAUUGUUAGCUGGU |
| 449 | 449a | |
| hsa-miR- | hsa-miR- | UAUGUGCCUUUGGACUACAUCG |
| 455 | 455-5p | |
| hsa-miR- | hsa-miR- | CAGCAGCACACUGUGGUUUGU |
| 497 | 497-5p | |
| hsa-miR- | hsa-miR-p | AAUCCUUUGUCCCUGGGUGAGA |
| 501 | 501-5 | |
| hsa-miR- | hsa-miR- | UCAGCAAACAUUUAUUGUGUGC |
| 545 | 545-3p | |
| hsa-miR- | hsa-miR- | AACAGGUGACUGGUUAGACAA |
| 552 | 552 | |
| hsa-miR- | hsa-miR- | UUGUGUCAAUAUGCGAUGAUGU |
| 592 | 592 | |
| hsa-miR- | hsa-miR- | UAUUCAUUUAUCCCCAGCCUACA |
| 664 | 664-3p | |

²TaqMan ® Human MicroRNA Assays, Applied Biosystems (www.appliedbiosystems.com, accessed January 6th 2013)
²miRBase 19, August 2012, www.mirbase.org (Homo sapiens)
³www.appliedbiosystems.com, accessed January 11th 2012

TABLE 9
Correlation between mean-normalized miRNA expression and outcome for CapOxBev treated patients

| | Discovery cohort ( [symbol] ) | | | | [symbol] | | | | [symbol] | | | |
|---|---|---|---|---|---|---|---|---|---|---|---|---|
| | TTP | | OS | | TTP | | OS | | TTP | | OS | |
| MiRNA name | Hazard ratio | p | Hazard ratio | P | Hazard ratio | p | Hazard ratio | p | Hazard ratio | p | Hazard ratio | p |
| [symbol] | | | | | 1.298 | 0.02609 | | | | | | |
| [symbol] | | | | | | | | | | | [symbol] | 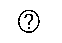 |
| [symbol] | 1.315 | [symbol] | | | 1.451 | 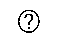 | | | 1.366 | 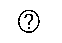 | 1.252 | 0.01513 |
| [symbol] | 1.374 | 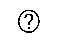 | 1.398 | [symbol] | | | | | 1.353 | 0.00029 | 1.370 | 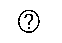 |
| [symbol] | | | | | | | | | | | [symbol] | [symbol] |
| [symbol] | [symbol] | [symbol] | 0.794 | 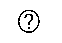 | | | | | | | | |
| [symbol] | | | | | 1.321 | [symbol] | | | | | | |
| [symbol] | | | | | | | | | | | 1.181 | [symbol] |
| [symbol] | [symbol] | 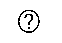 | 1.214 | 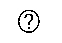 | | | 1.274 | [symbol] | | | 1.215 | 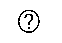 |
| [symbol] | | | 1.260 | [symbol] | | | 1.292 | [symbol] | | | [symbol] | [symbol] |
| [symbol] | | | | | | | | | | | 1.272 | 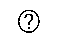 |
| [symbol] | | | | | | | | | [symbol] | [symbol] | | |
| [symbol] | | | [symbol] | [symbol] | [symbol] | [symbol] | [symbol] | [symbol] | [symbol] | [symbol] | [symbol] | [symbol] |
| [symbol] | | | [symbol] | [symbol] | [symbol] | [symbol] | [symbol] | 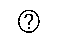 | 0.799 | [symbol] | 0.544 | 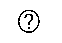 |

Hazard ratios and p-values are from univariate analysis. Hazard ratios are per inter-quartile range increase in 40-CT.
TTP, Time to disease progression,
OS, overall survival 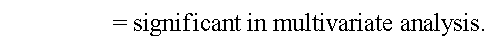 [symbol]
indicates data missing or illegible when filed

TABLE 10
MiRNAs showing trend (p < 0.1) for interaction between correlation
to outcome and bevacizumab treatment

| | TTP | OS |
|---|---|---|
| MiRNA name | p-ualue | p-value |
| miR-155-5p | 0.09 | |
| miR-185-5p | 0.06 | |
| miR-204-5p | 0.08 | |
| miR-214-5p | 0.07 | |
| miR-382-5p | 0.01 | |
| miR-455-5p | | 0.02 |
| miR-449a | | 0.06 |
| miR-664-3p | | 0.02 |

Mean-normalized expression values wore used.
TTP, Time to disease progression;
OS, overall survival.

[symbol]

TABLE 12
MiRNAs showing trend (p < 0.1) for interaction between correlation
to outcome and bevacizumab treatment

Primary tumor location

| | |
|---|---|
| Caecum to | Sigmoid colon |
| decending colon | and rectum |

| | TTP | OS | TTP | OS |
|---|---|---|---|---|
| MiRNA name | p-value | p-value | p-value | p-value |
| miR-17.3p | | | 0.04 | |
| miR-155.5p | 0.06 | 0.09 | | |
| miR-185.5p | 0.02 | 0.06 | 0.01 | 0.08 |
| miR-196b.5p | 0.06 | 0.02 | | |
| miR-204.5p | 0.03 | | | |
| miR-214.5p | 0.09 | 0.07 | | |
| miR-382.5p | | | 0.006 | |
| miR-455.5p | | | | 0.05 |
| miR-545.3p | | 0.09 | | |
| miR-664.3p | | | 0.07 | 0.02 |

TABLE 1
Univariately significant miRNAs for TTP using raw values.

| | | | 95% | |
|---|---|---|---|---|
| miRNA | HR | 95% Cl low | Cl high | p-value |
| miR-382 | 0.647 | 0.530 | 0.791 | <0.001 |
| miR-370 | 0.720 | 0.612 | 0.848 | <0.001 |
| miR-193b* | 0.620 | 0.489 | 0.787 | <0.001 |
| miR-22 | 0.800 | 0.713 | 0.898 | <0.001 |
| miR-497 | 0.759 | 0.658 | 0.877 | <0.001 |
| miR-29c* | 0.642 | 0.509 | 0.810 | <0.001 |
| miR-145* | 0.644 | 0.508 | 0.815 | <0.001 |
| miR-501-5p | 0.719 | 0.600 | 0.862 | <0.001 |
| miR-146b-3p | 0.651 | 0.513 | 0.828 | <0.001 |
| miR-29b | 0.710 | 0.586 | 0.860 | <0.001 |
| miR-185 | 0.745 | 0.629 | 0.882 | 0.001 |
| miR-17* | 0.742 | 0.624 | 0.881 | 0.001 |
| miR-34b | 0.726 | 0.602 | 0.876 | 0.001 |
| miR-423-5p | 0.721 | 0.595 | 0.874 | 0.001 |
| miR-576-3p | 0.737 | 0.616 | 0.883 | 0.001 |
| miR-214* | 0.720 | 0.590 | 0.877 | 0.001 |
| miR-874 | 0.775 | 0.664 | 0.904 | 0.001 |
| miR-190b | 0.803 | 0.703 | 0.918 | 0.001 |
| miR-152 | 0.721 | 0.586 | 0.886 | 0.002 |
| miR-324-3p | 0.785 | 0.671 | 0.919 | 0.003 |
| miR-99a | 0.744 | 0.614 | 0.903 | 0.003 |
| miR-204 | 0.785 | 0.666 | 0.924 | 0.004 |
| miR-455-5p | 0.776 | 0.654 | 0.921 | 0.004 |
| miR-143 | 0.754 | 0.621 | 0.915 | 0.004 |
| miR-505 | 0.761 | 0.629 | 0.920 | 0.005 |
| miR-660 | 0.798 | 0.682 | 0.935 | 0.005 |
| miR-34a | 0.816 | 0.708 | 0.942 | 0.005 |
| miR-29a* | 0.813 | 0.700 | 0.944 | 0.007 |
| miR-100 | 0.776 | 0.647 | 0.932 | 0.007 |
| miR-151-3p | 0.793 | 0.671 | 0.938 | 0.007 |
| miR-19b-1* | 0.740 | 0.595 | 0.921 | 0.007 |
| miR-29c | 0.780 | 0.650 | 0.937 | 0.008 |
| miR-1 | 0.754 | 0.612 | 0.929 | 0.008 |
| miR-125b | 0.777 | 0.644 | 0.938 | 0.009 |
| miR-196b | 0.773 | 0.638 | 0.937 | 0.009 |
| miR-101 | 0.784 | 0.653 | 0.940 | 0.009 |
| miR-452 | 0.784 | 0.653 | 0.941 | 0.009 |
| miR-338-3p | 0.809 | 0.690 | 0.949 | 0.009 |
| miR-579 | 0.801 | 0.678 | 0.947 | 0.010 |
| miR-218 | 0.776 | 0.641 | 0.940 | 0.010 |
| miR-28-5p | 0.797 | 0.671 | 0.947 | 0.010 |
| miR-125a-5p | 0.748 | 0.598 | 0.935 | 0.011 |
| miR-127-3p | 0.797 | 0.669 | 0.949 | 0.011 |
| miR-500 | 0.787 | 0.655 | 0.946 | 0.011 |
| miR-190 | 0.795 | 0.666 | 0.950 | 0.012 |
| miR-99a* | 0.833 | 0.722 | 0.960 | 0.012 |
| miR-18a | 0.769 | 0.627 | 0.944 | 0.012 |
| miR-365 | 0.749 | 0.598 | 0.938 | 0.012 |
| miR-539 | 0.760 | 0.613 | 0.943 | 0.012 |
| miR-26a-1* | 0.780 | 0.642 | 0.948 | 0.013 |
| miR-199b-5p | 0.763 | 0.617 | 0.945 | 0.013 |
| miR-217 | 0.746 | 0.592 | 0.941 | 0.013 |
| miR-15a | 0.816 | 0.695 | 0.959 | 0.013 |
| miR-141 | 0.787 | 0.650 | 0.952 | 0.014 |
| miR-296-5p | 0.788 | 0.650 | 0.955 | 0.015 |
| miR-133b | 0.726 | 0.560 | 0.940 | 0.015 |
| miR-548d-5p | 0.778 | 0.635 | 0.953 | 0.015 |
| miR-548c-5p | 0.778 | 0.634 | 0.954 | 0.016 |
| miR-130a | 0.827 | 0.708 | 0.965 | 0.016 |
| let.7i* | 0.738 | 0.576 | 0.946 | 0.017 |
| miR-142-3p | 0.802 | 0.669 | 0.961 | 0.017 |
| miR-141* | 0.789 | 0.649 | 0.958 | 0.017 |
| miR-639 | 0.775 | 0.628 | 0.956 | 0.017 |
| miR-379 | 0.843 | 0.733 | 0.971 | 0.018 |
| miR-187 | 0.747 | 0.587 | 0.950 | 0.018 |
| miR-361-5p | 0.825 | 0.704 | 0.967 | 0.018 |
| miR-134 | 0.757 | 0.601 | 0.953 | 0.018 |
| miR-487a | 0.785 | 0.642 | 0.961 | 0.019 |
| miR-145 | 0.783 | 0.637 | 0.963 | 0.020 |
| miR-128 | 0.795 | 0.655 | 0.966 | 0.021 |
| miR-769-5p | 0.845 | 0.732 | 0.975 | 0.021 |
| miR-592 | 0.773 | 0.621 | 0.962 | 0.021 |
| miR-543 | 0.785 | 0.638 | 0.965 | 0.022 |
| miR-451 | 0.819 | 0.689 | 0.972 | 0.023 |
| miR-27b | 0.834 | 0.714 | 0.975 | 0.023 |
| miR-21 | 0.805 | 0.667 | 0.971 | 0.023 |
| miR-340 | 0.843 | 0.727 | 0.978 | 0.024 |
| miR-20a* | 0.863 | 0.758 | 0.982 | 0.025 |
| miR-411* | 0.748 | 0.579 | 0.966 | 0.026 |
| miR-410 | 0.816 | 0.682 | 0.977 | 0.027 |
| miR-133a | 0.750 | 0.581 | 0.968 | 0.027 |
| miR-100* | 0.803 | 0.661 | 0.975 | 0.027 |
| miR-199a-5p | 0.783 | 0.630 | 0.973 | 0.027 |
| miR-99b | 0.804 | 0.661 | 0.977 | 0.029 |
| miR-92a | 0.821 | 0.687 | 0.981 | 0.030 |
| miR-369-5p | 0.802 | 0.657 | 0.979 | 0.030 |
| miR-210 | 0.794 | 0.643 | 0.980 | 0.032 |
| miR-362-5p | 0.834 | 0.705 | 0.985 | 0.033 |
| miR-193a-5p | 0.839 | 0.714 | 0.986 | 0.033 |
| miR-654-5p | 0.789 | 0.634 | 0.981 | 0.033 |
| miR-140-5p | 0.841 | 0.718 | 0.987 | 0.034 |
| miR-302c | 0.754 | 0.581 | 0.978 | 0.034 |
| miR-758 | 0.806 | 0.660 | 0.984 | 0.034 |
| miR-409-3p | 0.761 | 0.590 | 0.981 | 0.035 |
| miR-10a | 0.812 | 0.670 | 0.985 | 0.035 |
| miR-429 | 0.807 | 0.662 | 0.985 | 0.035 |
| miR-194* | 0.828 | 0.694 | 0.987 | 0.035 |
| miR-96 | 0.812 | 0.669 | 0.986 | 0.036 |
| miR-331-5p | 0.833 | 0.702 | 0.988 | 0.036 |
| miR-339-3p | 0.849 | 0.727 | 0.990 | 0.037 |
| miR-200b | 0.851 | 0.731 | 0.991 | 0.038 |
| miR-491-5p | 0.834 | 0.703 | 0.990 | 0.038 |
| miR-493 | 0.823 | 0.685 | 0.990 | 0.039 |
| miR-181c | 0.791 | 0.632 | 0.989 | 0.039 |
| miR-9* | 0.853 | 0.733 | 0.993 | 0.040 |
| miR-378 | 0.838 | 0.706 | 0.994 | 0.042 |
| miR-545 | 0.816 | 0.671 | 0.993 | 0.042 |
| miR-213 | 0.837 | 0.704 | 0.994 | 0.043 |
| miR-708 | 0.807 | 0.656 | 0.993 | 0.043 |
| miR-455-3p | 0.794 | 0.635 | 0.993 | 0.043 |
| miR-511 | 0.836 | 0.703 | 0.995 | 0.044 |
| miR-30a-5p | 0.860 | 0.741 | 0.998 | 0.047 |
| miR-376a | 0.804 | 0.648 | 0.998 | 0.048 |
| miR-490-3p | 0.814 | 0.663 | 0.999 | 0.049 |
| miR-532-5p | 0.841 | 0.707 | 0.999 | 0.049 |
| miR-1180 | 0.870 | 0.758 | 1.000 | 0.049 |

TABLE 2
Univariately significant miRNAs for OS using raw values.

| | | | 95% | |
|---|---|---|---|---|
| miRNA | HR | 95% Cl low | Cl high | p-value |
| miR-382 | 0.692 | 0.580 | 0.826 | <0.001 |
| miR-592 | 0.639 | 0.508 | 0.803 | <0.001 |
| miR-196b | 0.691 | 0.568 | 0.841 | <0.001 |
| miR-29b | 0.700 | 0.568 | 0.862 | 0.001 |
| miR-455-5p | 0.743 | 0.621 | 0.888 | 0.001 |
| miR-22 | 0.821 | 0.727 | 0.927 | 0.001 |
| miR-204 | 0.764 | 0.644 | 0.907 | 0.002 |
| miR-370 | 0.779 | 0.663 | 0.914 | 0.002 |
| miR-338-3p | 0.779 | 0.661 | 0.918 | 0.003 |
| miR-99a* | 0.794 | 0.682 | 0.924 | 0.003 |
| miR-133b | 0.662 | 0.504 | 0.870 | 0.003 |
| miR-15a | 0.769 | 0.645 | 0.917 | 0.003 |
| miR-497 | 0.793 | 0.675 | 0.932 | 0.005 |
| miR-29c* | 0.721 | 0.574 | 0.905 | 0.005 |
| miR-552 | 0.787 | 0.665 | 0.931 | 0.005 |
| miR-181a | 0.766 | 0.635 | 0.925 | 0.006 |
| miR-660 | 0.796 | 0.677 | 0.935 | 0.006 |
| miR-324-3p | 0.784 | 0.659 | 0.933 | 0.006 |
| miR-141 | 0.769 | 0.637 | 0.928 | 0.006 |
| miR-874 | 0.793 | 0.670 | 0.939 | 0.007 |
| miR-185 | 0.784 | 0.656 | 0.937 | 0.008 |
| miR-99a | 0.753 | 0.608 | 0.933 | 0.009 |
| miR-545 | 0.758 | 0.616 | 0.935 | 0.009 |
| miR-21* | 0.744 | 0.591 | 0.936 | 0.012 |
| miR-452 | 0.782 | 0.646 | 0.947 | 0.012 |
| miR-143 | 0.762 | 0.616 | 0.941 | 0.012 |
| miR-214* | 0.759 | 0.612 | 0.941 | 0.012 |
| miR-576-3p | 0.786 | 0.651 | 0.950 | 0.013 |
| miR-501-5p | 0.789 | 0.654 | 0.952 | 0.013 |
| miR-29c | 0.781 | 0.642 | 0.950 | 0.013 |
| miR-379 | 0.836 | 0.725 | 0.965 | 0.014 |
| miR-17* | 0.804 | 0.675 | 0.957 | 0.014 |
| miR-451 | 0.774 | 0.629 | 0.951 | 0.015 |
| miR-152 | 0.756 | 0.602 | 0.949 | 0.016 |
| miR-1825 | 0.744 | 0.586 | 0.946 | 0.016 |
| miR-193a-3p | 0.846 | 0.739 | 0.970 | 0.016 |
| miR-26a-1* | 0.774 | 0.628 | 0.954 | 0.017 |
| miR-1244 | 0.862 | 0.763 | 0.975 | 0.018 |
| miR-18b | 0.785 | 0.642 | 0.959 | 0.018 |
| miR-29a* | 0.820 | 0.695 | 0.968 | 0.019 |
| miR-337-5p | 0.779 | 0.632 | 0.961 | 0.020 |
| miR-106b | 0.808 | 0.675 | 0.966 | 0.020 |
| miR-148a | 0.791 | 0.649 | 0.963 | 0.020 |
| miR-339-3p | 0.834 | 0.713 | 0.974 | 0.022 |
| miR-361-5p | 0.812 | 0.679 | 0.973 | 0.024 |
| miR-491-5p | 0.799 | 0.658 | 0.971 | 0.024 |
| miR-532-5p | 0.806 | 0.669 | 0.972 | 0.024 |
| miR-181c* | 0.812 | 0.677 | 0.973 | 0.024 |
| miR-101 | 0.791 | 0.643 | 0.971 | 0.025 |
| miR-539 | 0.780 | 0.626 | 0.972 | 0.027 |
| miR-604 | 0.757 | 0.591 | 0.970 | 0.028 |
| miR-487a | 0.793 | 0.644 | 0.975 | 0.028 |
| miR-548c-5p | 0.787 | 0.636 | 0.974 | 0.028 |
| miR-145* | 0.740 | 0.564 | 0.969 | 0.029 |
| miR-30a-5p | 0.823 | 0.691 | 0.981 | 0.030 |
| miR-136* | 0.788 | 0.636 | 0.978 | 0.030 |
| miR-20a* | 0.854 | 0.741 | 0.985 | 0.030 |
| miR-548d-5p | 0.792 | 0.640 | 0.979 | 0.031 |
| miR-410 | 0.814 | 0.674 | 0.982 | 0.031 |
| miR-224 | 0.780 | 0.621 | 0.978 | 0.032 |
| miR-151-3p | 0.827 | 0.696 | 0.984 | 0.032 |
| miR-500 | 0.804 | 0.659 | 0.981 | 0.032 |
| miR-135b | 0.780 | 0.621 | 0.981 | 0.033 |
| miR-1247 | 0.746 | 0.569 | 0.977 | 0.033 |
| miR-449a | 1.256 | 1.018 | 1.551 | 0.034 |
| miR-572 | 0.805 | 0.657 | 0.986 | 0.036 |
| miR-28-5p | 0.803 | 0.652 | 0.989 | 0.039 |
| miR-590-5p | 0.866 | 0.755 | 0.994 | 0.041 |
| miR-7-2* | 1.335 | 1.010 | 1.765 | 0.042 |
| miR-133a | 0.746 | 0.561 | 0.992 | 0.044 |
| miR-19b | 0.827 | 0.687 | 0.995 | 0.044 |
| miR-627 | 0.829 | 0.690 | 0.996 | 0.045 |
| miR-213 | 0.827 | 0.685 | 0.997 | 0.046 |
| miR-140-5p | 0.830 | 0.690 | 0.998 | 0.047 |

TABLE 3
Univariately significant miRNAs for TTP using quantile normalized
values.

| | | | 95% | |
|---|---|---|---|---|
| miRNA | HR | 95% Cl low | Cl high | p-value |
| miR-145* | 0.645 | 0.513 | 0.811 | <0.001 |
| miR-185 | 0.820 | 0.733 | 0.917 | 0.001 |
| miR-22 | 0.808 | 0.716 | 0.912 | 0.001 |
| miR-497 | 0.644 | 0.499 | 0.832 | 0.001 |
| miR-193b* | 0.691 | 0.556 | 0.859 | 0.001 |
| miR-143 | 0.909 | 0.859 | 0.963 | 0.001 |
| miR-214* | 0.741 | 0.613 | 0.896 | 0.002 |
| miR-29b | 0.864 | 0.788 | 0.948 | 0.002 |
| miR-664 | 1.351 | 1.115 | 1.638 | 0.002 |
| miR-17* | 0.775 | 0.656 | 0.915 | 0.003 |
| miR-382 | 0.759 | 0.633 | 0.911 | 0.003 |
| miR-1285 | 1.447 | 1.129 | 1.856 | 0.004 |
| miR-204 | 0.745 | 0.607 | 0.914 | 0.005 |
| miR-155 | 1.447 | 1.112 | 1.882 | 0.006 |
| miR-532-3p | 1.097 | 1.025 | 1.173 | 0.008 |
| miR-1 | 0.703 | 0.541 | 0.913 | 0.008 |
| miR-146b-3p | 0.783 | 0.652 | 0.940 | 0.009 |
| miR-874 | 0.794 | 0.668 | 0.944 | 0.009 |
| miR-1227 | 1.145 | 1.033 | 1.269 | 0.010 |
| miR-29c* | 0.771 | 0.632 | 0.942 | 0.011 |
| miR-34b | 0.802 | 0.675 | 0.952 | 0.012 |
| miR-19b-1* | 0.832 | 0.720 | 0.960 | 0.012 |
| miR-100 | 0.767 | 0.623 | 0.943 | 0.012 |
| miR-576-3p | 0.739 | 0.583 | 0.937 | 0.012 |
| miR-365 | 0.697 | 0.525 | 0.926 | 0.013 |
| miR-660 | 0.863 | 0.768 | 0.970 | 0.014 |
| miR-145 | 0.752 | 0.598 | 0.946 | 0.015 |
| miR-505 | 0.797 | 0.662 | 0.958 | 0.016 |
| miR-501-5p | 0.772 | 0.626 | 0.953 | 0.016 |
| miR-625 | 1.156 | 1.025 | 1.304 | 0.018 |
| miR-579 | 0.799 | 0.660 | 0.967 | 0.021 |
| let-7i* | 0.814 | 0.681 | 0.974 | 0.024 |
| miR-191 | 1.212 | 1.023 | 1.435 | 0.026 |
| miR-28-5p | 0.899 | 0.818 | 0.988 | 0.027 |
| miR-452 | 0.793 | 0.646 | 0.974 | 0.027 |
| miR-340 | 0.774 | 0.611 | 0.980 | 0.033 |
| miR-101 | 0.806 | 0.660 | 0.983 | 0.034 |
| miR-29a* | 0.861 | 0.749 | 0.990 | 0.036 |
| miR-199a-3p | 0.831 | 0.698 | 0.988 | 0.037 |
| miR-338-3p | 0.811 | 0.665 | 0.988 | 0.038 |
| miR-16 | 1.064 | 1.004 | 1.128 | 0.038 |
| miR-18a | 0.740 | 0.555 | 0.985 | 0.039 |
| miR-15a | 0.798 | 0.642 | 0.991 | 0.041 |
| miR-26a-1* | 0.816 | 0.670 | 0.992 | 0.042 |
| miR-197 | 1.165 | 1.006 | 1.351 | 0.042 |
| miR-190b | 0.871 | 0.761 | 0.997 | 0.045 |
| miR-99a | 0.774 | 0.602 | 0.994 | 0.045 |
| miR-125b | 0.814 | 0.665 | 0.996 | 0.046 |
| miR-133b | 0.755 | 0.573 | 0.996 | 0.047 |
| miR-96 | 0.812 | 0.660 | 0.998 | 0.048 |
| miR-455-5p | 0.892 | 0.795 | 1.000 | 0.050 |

TABLE 4
Univariately significant miRNAs for OS using quantile normalized values

| | | | 95% | |
|---|---|---|---|---|
| miRNA | HR | 95% Cl low | Cl high | p-value |
| miR-196b | 0.581 | 0.446 | 0.758 | <0.001 |
| miR-592 | 0.584 | 0.448 | 0.761 | <0.001 |
| miR-185 | 0.816 | 0.730 | 0.913 | <0.001 |
| miR-545 | 0.643 | 0.501 | 0.827 | 0.001 |
| miR-29b | 0.848 | 0.772 | 0.932 | 0.001 |
| miR-204 | 0.688 | 0.554 | 0.854 | 0.001 |
| miR-15a | 0.679 | 0.541 | 0.852 | 0.001 |
| miR-455-5p | 0.827 | 0.740 | 0.925 | 0.001 |
| miR-22 | 0.817 | 0.724 | 0.923 | 0.001 |
| miR-338-3p | 0.718 | 0.588 | 0.878 | 0.001 |
| miR-19b | 0.725 | 0.597 | 0.882 | 0.001 |
| miR-143 | 0.905 | 0.850 | 0.963 | 0.002 |
| miR-382 | 0.753 | 0.629 | 0.900 | 0.002 |
| miR-660 | 0.860 | 0.779 | 0.950 | 0.003 |
| miR-148a | 0.782 | 0.664 | 0.921 | 0.003 |
| miR-155 | 1.523 | 1.150 | 2.018 | 0.003 |
| miR-449a | 1.472 | 1.132 | 1.915 | 0.004 |
| miR-106b | 0.721 | 0.578 | 0.901 | 0.004 |
| miR-141 | 0.656 | 0.492 | 0.876 | 0.004 |
| miR-18b | 0.794 | 0.676 | 0.931 | 0.005 |
| miR-379 | 0.725 | 0.576 | 0.912 | 0.006 |
| miR-214* | 0.756 | 0.618 | 0.923 | 0.006 |
| miR-552 | 0.745 | 0.604 | 0.920 | 0.006 |
| miR-29c | 0.645 | 0.470 | 0.885 | 0.007 |
| miR-1227 | 1.161 | 1.042 | 1.293 | 0.007 |
| miR-625 | 1.238 | 1.060 | 1.446 | 0.007 |
| miR-181a | 0.772 | 0.639 | 0.933 | 0.007 |
| miR-193a-3p | 0.859 | 0.769 | 0.960 | 0.007 |
| miR-497 | 0.684 | 0.517 | 0.907 | 0.008 |
| miR-636 | 1.232 | 1.055 | 1.437 | 0.008 |
| miR-133b | 0.675 | 0.504 | 0.905 | 0.009 |
| miR-449b | 1.389 | 1.084 | 1.781 | 0.009 |
| miR-596 | 1.391 | 1.082 | 1.788 | 0.010 |
| miR-17* | 0.801 | 0.676 | 0.950 | 0.011 |
| miR-452 | 0.760 | 0.614 | 0.941 | 0.012 |
| miR-29c* | 0.764 | 0.619 | 0.942 | 0.012 |
| miR-362-3p | 1.065 | 1.014 | 1.119 | 0.012 |
| miR-136* | 0.720 | 0.557 | 0.931 | 0.012 |
| miR-145* | 0.725 | 0.564 | 0.932 | 0.012 |
| miR-576-3p | 0.724 | 0.562 | 0.933 | 0.012 |
| miR-1825 | 0.720 | 0.553 | 0.937 | 0.014 |
| miR-28-5p | 0.859 | 0.760 | 0.971 | 0.015 |
| miR-20a* | 0.802 | 0.670 | 0.960 | 0.016 |
| miR-451 | 0.849 | 0.741 | 0.973 | 0.019 |
| miR-7-2* | 1.441 | 1.062 | 1.955 | 0.019 |
| miR-26a-1* | 0.770 | 0.618 | 0.959 | 0.019 |
| miR-29a* | 0.836 | 0.718 | 0.973 | 0.021 |
| miR-99a* | 0.862 | 0.760 | 0.978 | 0.021 |
| miR-181c* | 0.894 | 0.813 | 0.983 | 0.021 |
| miR-19b-1* | 0.848 | 0.736 | 0.976 | 0.021 |
| miR-101 | 0.764 | 0.607 | 0.961 | 0.021 |
| miR-627 | 0.835 | 0.714 | 0.976 | 0.023 |
| miR-642 | 1.287 | 1.032 | 1.605 | 0.025 |
| miR-520b | 1.168 | 1.020 | 1.337 | 0.025 |
| miR-152 | 0.757 | 0.594 | 0.966 | 0.025 |
| miR-532-3p | 1.094 | 1.011 | 1.184 | 0.026 |
| miR-874 | 0.804 | 0.662 | 0.975 | 0.026 |
| miR-224 | 0.791 | 0.641 | 0.977 | 0.030 |
| miR-491-5p | 0.781 | 0.624 | 0.978 | 0.032 |
| miR-410 | 0.762 | 0.593 | 0.978 | 0.033 |
| miR-200a | 0.722 | 0.532 | 0.982 | 0.038 |
| miR-652 | 0.850 | 0.729 | 0.992 | 0.039 |
| miR-361-5p | 0.855 | 0.737 | 0.992 | 0.039 |
| miR-331-3p | 1.352 | 1.014 | 1.804 | 0.040 |
| miR-1243 | 1.280 | 1.010 | 1.623 | 0.041 |
| miR-649 | 0.791 | 0.630 | 0.993 | 0.043 |
| miR-502-3p | 0.820 | 0.677 | 0.994 | 0.044 |
| miR-886-3p | 1.340 | 1.008 | 1.781 | 0.044 |
| miR-548d-5p | 0.831 | 0.692 | 0.998 | 0.048 |
| miR-487a | 0.800 | 0.640 | 0.999 | 0.049 |

TABLE 7
Mature sequence of miRNAs
from multivariate analyses

| miRNA | | |
|---|---|---|
| name | | |
| from | miRBase | mature |
| array¹ | name² | sequence³ |
| miR-1 | hsa-miR-1 | UGGAAUGUAAAGAAGUAUGUAU |
| miR-15a | hsa-miR- | UAGCAGCACAUAAUGGUUUGUG |
| | 15a-5p | |
| miR-17* | hsa-miR- | ACUGCAGUGAAGGCACUUGUAG |
| | 17-3p | |
| miR-22 | hsa-miR- | AAGCUGCCAGUUGAAGAACUGU |
| | 22-3p | |
| miR-29a* | hsa-miR- | ACUGAUUUCUUUUGGUGUUCAG |
| | 29a-5p | |
| miR-29b | hsa-miR- | UAGCACCAUUUGAAAUCAGUGUU |
| | 29b-3p | |
| miR-145* | hsa-miR- | GGAUUCCUGGAAAUACUGUUCU |
| | 145-3p | |
| miR-148a | hsa-miR- | UCAGUGCACUACAGAACUUUGU |
| | 148a-3p | |
| miR-155 | hsa-miR- | UUAAUGCUAAUCGUGAUAGGGGU |
| | 155-5p | |
| miR-181a | hsa-miR- | AACAUUCAACGCUGUCGGUGAGU |
| | 181a-5p | |
| miR-185 | hsa-miR- | UGGAGAGAAAGGCAGUUCCUGA |
| | 185-5p | |
| miR-193b* | hsa-miR- | CGGGGUUUUGAGGGCGAGAUGA |
| | 193b-5p | |
| miR-196b | hsa-miR- | UAGGUAGUUUCCUGUUGUUGGG |
| | 196b-5p | |
| miR-204 | hsa-miR- | UUCCCUUUGUCAUCCUAUGCCU |
| | 204-5p | |
| miR-214* | hsa-miR- | UGCCUGUCUACACUUGCUGUGC |
| | 214-5p | |
| miR-338- | hsa-miR- | UCCAGCAUCAGUGAUUUUGUUG |
| 3p | 338-3p | |
| miR-365 | hsa-miR- | UAAUGCCCCUAAAAAUCCUUAU |
| | 365a-3p | |
| miR-382 | hsa-miR- | GAAGUUGUUCGUGGUGGAUUCG |
| | 382-5p | |
| miR-449a | hsa-miR- | UGGCAGUGUAUUGUUAGCUGGU |
| | 449a | |
| miR-449b | hsa-miR- | AGGCAGUGUAUUGUUAGCUGGC |
| | 449b-5p | |
| miR-455- | hsa-miR- | UAUGUGCCUUUGGACUACAUCG |
| 5p | 455-5p | |
| miR-497 | hsa-miR- | CAGCAGCACACUGUGGUUUGU |
| | 497-5p | |
| miR-501- | hsa-miR- | AAUCCUUUGUCCCUGGGUGAGA |
| 5p | 501-5p | |
| miR-545 | hsa-miR- | UCAGCAAACAUUUAUUGUGUGC |
| | 545-3p | |
| miR-552 | hsa-miR- | AACAGGUGACUGGUUAGACAA |
| | 552 | |
| miR-592 | hsa-miR- | UUGUGUCAAUAUGCGAUGAUGU |
| | 592 | |
| miR-664 | hsa-miR- | UAUUCAUUUAUCCCCAGCCUACA |
| | 664-3p | |
| miR-1251 | hsa-miR- | ACUCUAGCUGCCAAAGGCGCU |
| | 1251 | |

¹TadManHuman ® MicroRNA A Cards v2.0 and B Cards v3.0 (Part Number 4400238, Applied Biosystems)
²miRBase 18, November 2011, www.mirbase.org (Homo sapiens), accessed January 11th 2012
³www.appliedbiosystems.com, accessed January 11th 2012

### REFERENCES

1. Andersen P K, Gill R D. Cox Regression-Model for Counting-Processes—a Large Sample Study. Ann Stat. 1982; 10(4):1100-20.
2. Cox D R. Regression Models and Life-Tables. J Roy Stat Soc B. 1972; 34(2):187-&.
3. Sakamoto MIGKY. Akaike Information Criterion Statistics: D. Reidel Publishing Company; 1986.
4. Birnbaum Z W, Tingey F H. One-Sided Confidence Contours for Probability Distribution Functions. Ann Math Stat. 1951; 22(4):592-6.
5. Conover W J. Practical Nonparametric Statistics: New York: John Wiley & Sons.; 1971.
6. Durbin J. Distribution Theory for Tests Based on the Sample Distribution Function: Society for Industrial Mathematics; 1987.
7. Marsaglia W W T J W G. Evaluating Kolmogorov's Distribution. Journal of Statistical Software. 203 Nov. 2003; 8(18).
8. Bovelstad H M, Nygard S, Storvold H L, Aldrin M, Borgan O, Frigessi A, et al. Predicting survival from microarray data—a comparative study. Bioinformatics. 2007 Aug. 15; 23(16):2080-7.
9. Bolstad B M, Irizarry R A, Astrand M, Speed T P. A comparison of normalization methods for high density oligonucleotide array data based on variance and bias. Bioinformatics. 2003 Jan. 22; 19(2):185-93.
10. Boisen M K, Johansen J S, Dehlendorff C, Larsen J S, Østerlind K, Hansen J, Nielsen S E, Pfeiffer P, Tarpgaard L S, Hollander N H, Keldsen N, Hansen T F, Jensen B B, Jensen B V. Outcome for patients with metastatic colorectal cancer treated with first line capecitabine and oxaliplatin with or without bevacizumab according to location of the primary tumor. Submitted.

1-106. (canceled)

107. A method for predicting the efficacy of an anti-angiogenic treatment, alone or in combination with chemotherapy, in an individual suffering from cancer, said method comprising the steps of:
i) providing a sample comprising cancer cells from said individual; and
ii) determining the expression level of a combination of miRNAs in said sample, wherein the combination of miRNAs comprises at least two miRNAs; said combination comprising:
a) miR-664 and/or miR-455; and optionally one or more miRNAs selected from the group consisting of miR-1, miR-15a, miR-17*, miR-22, miR-29b, miR-145*, miR-155, miR-185, miR-193b*, miR-196b, miR-204, miR-214*, miR-338-3p, miR-382, miR-449, miR-455, miR-497, miR-501, miR-545, miR-552 and miR-592, or
b) at least one miRNA selected from the group consisting of miR-145*, miR-185, miR-22, miR-497, miR-193b*, miR-143, miR-214*, miR-29b, miR-664, miR-17*, miR-382, miR-1285, miR-204, miR-155, miR-532-3p, miR-1, miR-146 b-3p, miR-874, miR-1227, miR-29c*, miR-34b, miR-19b-1*, miR-100, miR-576-3p, miR-365, miR-660, miR-145, miR-505, miR-501-5p and miR-625; and at least one miRNA selected from the group consisting of miR-196b, miR-592, miR-545, miR-15a, miR-455-5p, miR-338-3p, miR-19b, miR-148a, miR-449a, miR-106b, miR-141, miR-18b, miR-379, miR-552, miR-29c, miR-181a, miR-193a-3p and miR-636,
wherein said individual has been subjected to anti-angiogenic treatment or is at risk of becoming subjected to anti-angiogenic treatment,
wherein the miRNA expression level, and/or an aberrant miRNA expression level, of at least one of said miRNAs is indicative of the efficacy of an anti-angiogenic treatment of said individual.

108. The method according to claim 107, said method further comprising one or more steps of:
a) extracting RNA from said sample,
b) comparing the expression level of at least one miRNA in said sample with a predetermined control level of the miRNA, wherein a difference in expression level is considered aberrant expression of said miRNA in said sample,
c) determining the difference in expression level between said sample and said predetermined control level, and/or
d) extracting the sample by fine-needle aspiration, by coarse-needle aspiration or by colorectal surgery.

109. The method according to claim 108, wherein the predetermined control level is i) the expression level of said miRNA in a control sample obtained from a patient suffering from cancer, wherein the patient has a good efficacy of an anti-angiogenic treatment or ii) the average of the expression level of said miRNA in at least 25 control samples from patients suffering from cancer, wherein the patients each have good efficacy of an anti-angiogenic treatment.

110. The method according to claim 109, wherein no or little difference in expression level is indicative of a good efficacy of an anti-angiogenic treatment in said individual.

111. The method according to claim 110, wherein no or little difference in expression level is evaluated using the Ct-value, wherein a difference in Ct-values between the Ct value of said sample and the predetermined control Ct value is considered close to the predetermined control level if the difference in Ct-values are of a value selected from the group consisting of less than 5, less than 4, less than 3, and less than 2.

112. The method according to claim 108, wherein the predetermined control level is i) the expression level of said miRNA in a control sample obtained from a patient suffering from cancer, wherein the patient has no or little efficacy of an anti-angiogenic treatment or ii) the average of the expression level in at least 25 different control samples from patients suffering from cancer, wherein the patients have no or little efficacy of an anti-angiogenic treatment.

113. The method according to claim 112, wherein a difference in expression level is indicative of a good efficacy of an anti-angiogenic treatment in said individual.

114. The method according to claim 107, wherein the cancer is selected from the group consisting of colorectal cancer, lung cancer, breast cancer, ovarian cancer, kidney cancer, pancreatic cancer and glioblastoma.

115. The method according to claim 114, wherein the colorectal cancer is selected from the group consisting of cancers of the colon, cancers of the rectum and cancers of the appendix; adenocarcinoma of the colon, the rectum or the appendix; colorectal cancer with a primary tumor in the sigmoid colon, rectum and/or rect-sigmoid colon; a colorectal cancer with one or more metastasis to the liver, lungs, brain, bone, peritoneum, and/or lymph nodes.

116. The method according to claim 107, wherein said sample is selected from the group consisting of a tissue sample; a tissue sample of the colon, the rectum or the appendix; a cancer tissue sample of the sigmoid colon, the rectum or the recto-sigmoid colon; a cancer tissue sample of a primary tumor originating in the sigmoid colon, the rectum or the recto-sigmoid colon; a tissue sample comprising colorectal carcinoma cells; and a blood sample.

117. The method according to claim 107, wherein said anti-angiogenic treatment is selected from the group consisting of an inhibitor of VEGF; an inhibitor of a VEGF receptor; an antibody or antigen binding fragment thereof that specifically binds to VEGF; and an antibody or antigen binding fragment thereof that specifically binds to a VEGF receptor.

118. The method according to claim 117, wherein said anti-angiogenic treatment is selected from the group consisting of bevacizumab, ranibizumab, lenvatinib, motesanib, pazopanib, lapatinib, sunitinib, sorafenib, axitinib, aflibercept and regorafenib.

119. The method according to claim 107, wherein said combination of miRNAs comprises two or more of i) miR-1, miR-17*, miR-22, miR-29a*, miR-29b, miR-145*, miR-185, miR-193b*, miR-204, miR-214*, miR-365, miR-382, miR-497, miR-501-5p, miR-664 or miR-1251; and/or ii) miR-15a, miR-22, miR-29b, miR-148a, miR-155, miR-181a, miR-196b, miR-204, miR-214*, miR-338-3p, miR-382, miR-449a, miR-449 b, miR-455-p, miR-497, miR-545, miR-552 or miR-592.

120. The method according to claim 107, wherein the expression level is determined of at least:
i) miR-664 and wherein a high expression level of miR-664 is indicative of enhanced efficacy of anti-angiogenic treatment;
ii) miR-22, and wherein a low expression level of miR-22 is indicative of enhanced efficacy of anti-angiogenic treatment.
iii) miR-145* and wherein a low expression level of miR-145* is indicative of enhanced efficacy of anti-angiogenic treatment
iv) miR-196b and wherein a high expression level of miR-196b is indicative of enhanced efficacy of anti-angiogenic treatment
v) miR-455 and wherein a low expression level of miR-455 is indicative of enhanced efficacy of anti-angiogenic treatment, and/or
vi) one or more of miR-17*, miR-145, miR-155, miR-185, miR-204, miR-214*, miR-382, miR-449, miR-501, miR-545, miR-552, miR-592.

121. The method according to claim 107, wherein the expression level of the combination of miRNAs is determined by microarray technique, quantitative polymerase chain reaction (QPCR), northern blot, nuclease protection assay, or in situ hybridization.

122. A method for treatment of cancer in an individual in need thereof, said method comprising the steps of:
a) predicting the efficacy of an anti-angiogenic treatment in said individual by the method according to claim 107, and
b) administering a therapeutically effective amount of an anti-angiogenic treatment and/or a chemotherapeutic treatment to said individual, provided the prediction of said efficacy is good,
thereby treating cancer in said individual.

123. A device for measuring the expression level of two or more miRNAs in a sample, wherein said device comprises one or more probes or one or more primer sets for two or more miRNAs, wherein said probes or primer sets consist of one or more probes or primer sets for two or more of the miRNAs according to claim 107.

124. The device according to claim 123, wherein said device comprises at least one probe or primer set for miR-664 and/or at least one probe or primer set for miR-455.

125. The device according to claim 123, wherein said device is selected from the group consisting of a microarray chip; an array chip; a microarray chip comprising DNA probes; a microarray chip comprising antisense miRNA probes; a QPCR Microfluidic Card; QPCR tubes; QPCR tubes in a strip; a QPCR plate; probes on a solid support; probes on at least one bead; and probes in liquid form in a tube.


SEQUENCE LISTING (22 sequences)

SEQ ID NO: 1 - RNA, 22 nt, artificial sequence
  miRNA sequence
uggaauguaaagaaguauguau

SEQ ID NO: 2 - RNA, 22 nt, artificial sequence
  miRNA sequence
uagcagcacauaaugguuugug

SEQ ID NO: 3 - RNA, 22 nt, artificial sequence
  miRNA sequence
acugcagugaaggcacuuguag

SEQ ID NO: 4 - RNA, 22 nt, artificial sequence
  miRNA sequence
aagcugccaguugaagaacugu

SEQ ID NO: 5 - RNA, 23 nt, artificial sequence
  miRNA sequence
uagcaccauuugaaaucaguguu

SEQ ID NO: 6 - RNA, 22 nt, artificial sequence
  miRNA sequence
ggauuccuggaaauacuguucu

SEQ ID NO: 7 - RNA, 23 nt, artificial sequence
  miRNA sequence
uuaaugcuaaucgugauaggggu

SEQ ID NO: 8 - RNA, 22 nt, artificial sequence
  miRNA sequence
uggagagaaaggcaguuccuga

SEQ ID NO: 9 - RNA, 22 nt, artificial sequence
  miRNA sequence
cgggguuuugagggcgagauga

SEQ ID NO: 10 - RNA, 22 nt, artificial sequence
  miRNA sequence
uagguaguuuccuguuguuggg

SEQ ID NO: 11 - RNA, 22 nt, artificial sequence
  miRNA sequence
uucccuuugucauccuaugccu

SEQ ID NO: 12 - RNA, 22 nt, artificial sequence
  miRNA sequence
ugccugucuacacuugcugugc

SEQ ID NO: 13 - RNA, 22 nt, artificial sequence
  miRNA sequence
uccagcaucagugauuuuguug

SEQ ID NO: 14 - RNA, 22 nt, artificial sequence
  miRNA sequence
gaaguuguucgugguggauucg

SEQ ID NO: 15 - RNA, 22 nt, artificial sequence
  miRNA sequence
uggcaguguauuguuagcuggu

SEQ ID NO: 16 - RNA, 22 nt, artificial sequence
  miRNA sequence
uaugugccuuuggacuacaucg

SEQ ID NO: 17 - RNA, 21 nt, artificial sequence
  miRNA sequence
cagcagcacacugugguuugu

SEQ ID NO: 18 - RNA, 22 nt, artificial sequence
  miRNA sequence
aauccuuugucccugggugaga

SEQ ID NO: 19 - RNA, 22 nt, artificial sequence
  miRNA sequence
ucagcaaacauuuauugugugc

SEQ ID NO: 20 - RNA, 21 nt, artificial sequence
  miRNA sequence
aacaggugacugguuagacaa

SEQ ID NO: 21 - RNA, 22 nt, artificial sequence
  miRNA sequence
uugugucaauaugcgaugaugu

SEQ ID NO: 22 - RNA, 23 nt, artificial sequence
  miRNA sequence
uauucauuuauccccagccuaca